(19) United States
(12) Patent Application Publication
(10) Pub. No.: US 2009/0099175 A1
(43) Pub. Date: Apr. 16, 2009

(54) Phosphodiesterase 10 inhibitors

(76) Inventors: Mark P. Arrington, Westwood, NJ (US); Allen T. Hopper, Mahwa, NJ (US); Richard D. Conticello, Cortlandt Manor, NY (US); Truc M. Nguyen, Des Moines, IA (US); Hans-Jurgen E. Hess, Old Lyme, CT (US); Carla Maria Gauss, White Plains, NY (US); Stephen A. Hitchcock, Westlake Village, CA (US)

Correspondence Address: JONES DAY, 222 EAST 41ST ST, NEW YORK, NY, 10017, US

(21) Appl. No.: 11/713,234

(22) Filed: Feb. 28, 2007

Related U.S. Application Data
(60) Provisional application No. 60/778,590, filed on Mar. 1, 2006.

(51) Int. Cl. A61K 31/502; C07D 237/28; C07D 237/30; C07D 471/04; C07D 401/04; A61K 31/506; A61K 31/5377; C07D 403/04; C07D 401/14; C07D 413/04; A61P 25/00

(52) U.S. Cl. 514/234.5; 544/235; 544/237; 544/116; 514/248; 514/266.21

(57) ABSTRACT

The present invention if directed to certain cinnoline compounds that are PDE10 inhibitors, pharmaceutical compositions containing such compounds and processes for preparing such compounds. The invention is also directed to methods of treating diseases mediated by PDE10 enzyme, such as obesity, non-insulin dependent diabetes, schizophrenia, bipolar disorder, obsessive-compulsive disorder, and the like.

### CROSS-REFERENCE

This application claims the benefit of U.S. Provisional Application No. 60/778,590, filed Mar. 1, 2006, the disclosure of which is incorporated herein by reference in its entirety.

### FIELD OF THE INVENTION

The present invention is directed to certain cinnoline compounds that are PDE10 inhibitors, pharmaceutical compositions containing such compounds and processes for preparing such compounds. This invention is also directed to methods of treating disorders or diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE1 is stimulated by Ca²⁺/calmodulin. PDE2 activity is stimulated by cGMP. PDE3 is inhibited by cGMP. PDE4 is cAMP specific and is specifically inhibited by rolipram. PDE5 is cGMP-specific. PDE6 is expressed in retina.

PDE10 sequences were identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least three alternative exons encode N termini and two exons encode C-termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunoprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, for example, in neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein is a compound of Formula (I):

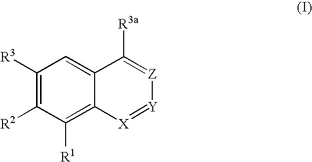

or an individual stereoisomer, a mixture of stereoisomers, or a pharmaceutically acceptable salt thereof, wherein:
Y and Z are nitrogen and X is —CR═ (where R is hydrogen, alkyl, cyano, or halo); or X and Y are nitrogen and Z is ═CH—; or X and Z are nitrogen and Y is ═CH—;
R¹, R², and R³ are independently hydrogen, alkyl, alkoxy, halo, haloalkyl, haloalkoxy, cyano, hydroxy, carboxy, alkoxycarbonyl, amino, alkylamino, dialkylamino, alkylcarbonyl, or cycloalkyl;
provided that at least one of R¹, R², and R³ is not hydrogen, and provided that when X and Y or X and Z are nitrogen and R¹ is hydrogen, then R² and R³ are not both independently hydroxy, alkoxy, or haloalkoxy; and
R^{3a} is an aryl, heteroaryl, or heterocyclyl ring substituted with:
   R⁴, wherein R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶, where R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; provided that at least one of R⁴, R⁵ and R⁶ is not hydrogen;
   wherein the aromatic or alicyclic ring in R⁴, R⁵, R⁶, and R⁷ is optionally substituted with one to three substituents independently selected from R^{a}, R^{b}, and R^{c}, which each are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e}, wherein R^{d} and R^{e} are chloro or fluoro;
provided that:

(i) when R^{3a} is pyrrolidin-1-yl, then R⁴ is not —X¹R⁷, where X¹ is —O— and R⁷ is substituted or unsubstituted aryl or heteroaryl;

(ii) when X and Y or X and Z are nitrogen, R^{3a} is piperidin-1-yl, one of R⁵ and R⁶ is hydrogen, and R⁴ is substituted or unsubstituted aryl or heteroaryl, then the other of R⁵ and R⁶ is not hydrogen, alkyl, carboxy, alkoxycarbonyl, cyano, hydroxyl, alkoxy, —COR, —CONRR′ or —NRR′ (where R and R′ are independently hydrogen, alkyl, or unsubstituted aryl), or —NHCOR (where R is alkyl or unsubstituted aryl);

(iii) when X and Y or X and Z are nitrogen, R^{3a} is piperidin-1-yl, both of R⁵ and R⁶ are hydrogen, or one of R⁵ and R⁶ is hydrogen and the other of R⁵ and R⁶ is substituted or unsubstituted aryl or heteroaryl, then R⁴ is not hydrogen, alkyl, —COR⁷ (where R⁷ is unsubstituted aryl), —COOR⁷ (where R⁷ is unsubstituted aryl), —CONR⁷R⁹, —NR⁷R¹⁰, or —NHCOR⁷ (where R⁹ and R¹⁰ are hydrogen, alkyl, or unsubstituted aryl; and each R⁷ is unsubstituted aryl);

(iv) when X and Y are nitrogen, two of R¹, R² and R³ are hydrogen and the other of R¹, R², and R³ is alkyl or halo, and R^{3a} is aryl, then (a) when two of R⁴, R⁵ and R⁶ are hydrogen, then the other of R⁴, R⁵ and R⁶ is not alkyl, halo, hydroxy, —COR′ (where R′ is alkyl) or —OC(O)R′, or —SO₂R′ (where R′ is aryl optionally substituted with alkyl); and (b) when one of R⁴, R⁵ and R⁶ is hydrogen, then the other of R⁴, R⁵ and R⁶ are not independently selected from alkyl, hydroxy, or —OCOR′ (where R′ is aryl);

(v) when X and Y are nitrogen, two of R¹, R² and R³ are hydrogen and the other of R¹, R², and R³ is halo, then R^{3a} is not indolin-1-yl or indol-1-yl, each substituted with alkyl and alkoxy and a third substituent selected from —CH₂—C(O)—OR′, wherein R′ is hydrogen or methyl;

(vi) when X and Z or Y and Z are nitrogen, then R^{3a} is not
(a) substituted or unsubstituted 1,2,3,4-tetrahydroquinolinyl;
(b) indolin-1-yl substituted with R⁴, R⁵ and R⁶, where two of R⁴, R⁵ and R⁶ are hydrogen and the other of R⁴, R⁵ and R⁶ is halo;
(c) piperidin-1-yl substituted with R⁴, R⁵ and R⁶, where two of R⁴, R⁵ and R⁶ are hydrogen and the other of R⁴, R⁵ and R⁶ is
   quinazoline-2,4(1H,3H)-dione or quinazolin-4(3H)-one each of which is optionally substituted with one or two substituents independently selected from nitro and alkyl;
   hydroxy, hydroxyalkyl, hydroxyalkyloxy, alkyl, carboxy, alkoxy, alkoxyalkyl, alkoxyalkyloxy, —COR [where R is aryl substituted with one halo], -(alkylene)-NRR′ [where R is hydrogen or —COR^{a} (where R^{a} is alkyl), and R′ is hydrogen or alkyl], —O-(alkylene)-NRR′ [where R is hydrogen or —COR^{a} (where R^{a} is alkyl), and R′ is hydrogen or alkyl], —NRR′ [where R is hydrogen or alkyl, and R′ is alkyl, —COR″ (where R″ is alkyl, haloalkyl, or aryl), —SO₂R″ (where R″ is pyridinyl, aralkyl, alkyl, cycloalkyl, or aryl optionally substituted with two alkoxy groups)], piperidin-4-yl-alkyl, piperidin-4-yl, or piperazin-4-yl-alkyl (wherein the piperidinyl in piperidin-4-yl-alkyl or piperidin-4-yl and piperazinyl in piperazin-4-yl-alkyl is substituted with a quinazoline ring optionally substituted with one to three substituents selected from halo, alkyl, alkoxy, haloalkyl, amino, monoalkylamino, or dialkylamino);
   2-oxoimidazolidin-1-yl, pyrrolidine-2,5-dione, or 1H-benzo[d]imidazol-2(3H)-one, optionally substituted with one alkyl; or
   furanylalkyloxy, 3,4-dihydroquinazolin-2(1H)-one, 1,6-alkylquinazoline-2,4(1H,3H)-dione, 1H-benzo[d][1,2,3]triazole, 3,4-dihydrobenzo[e][1,3]oxazin-2-one, 2H-pyran-2-ylalkyloxy, or tetrahydropyrimidin-2(1H)-one-1-ylalkyl, each of which is optionally substituted with alkyl;
(d) imidazolidin-2-one, optionally substituted with one alkyl;
(e) piperidin-1-yl, where one of R⁴, R⁵, and R⁶ is hydrogen; the other of R⁴, R⁵, and R⁶ is hydroxyl, and the third of R⁴, R⁵, and R⁶ is alkyl, aralkyl, or aryl optionally substituted with one or two substitutents independently selected from halo, hydroxyl, or alkoxy;
(f) indol-1-yl substituted with alkyl and alkoxy, and a third substituent selected from alkoxycarbonyl or hydroxyalkyl;
(g) aryl substituted with one or two substitutents independently selected from alkoxy, hydroxyl, alkyl, haloalkyl, acetyl, or 4-methylphenylsulfonyl;
(h) piperazin-1-yl substituted with R⁴, R⁵ and R⁶, wherein two of R⁴, R⁵ and R⁶ are hydrogen, and the other of R⁴, R⁵ and R⁶ is acyl; alkyl; aryl optionally substituted with one halo; alkoxycarbonyl; or —CONHR′ (where R′ is aryl optionally substituted with hydroxyl, cyano, nitro, alkyl, or alkylcarbonyl); or morpholin-4-ylcarbonyl;
(i) aryl substituted with R⁴, R⁵, and R⁶, where R⁵ is hydrogen and one of R⁴ and R⁶ is alkyl, halo, amino, nitro, hydroxyl, alkoxy, phenyl, haloalkyl, dialkylamino, or —NHCOR′ (where R′ is alkyl), and the other of R⁴ and R⁶ is hydrogen, alkyl, amino, or alkoxy; or all R⁴, R⁵, R⁶ are alkoxy; or
(j) 3-halopyridin-4-yl;

(vii) when X and Z or Y and Z are nitrogen, then when two of R¹, R², and R³ are hydrogen, then the other of R¹, R², and R³ is not halo;

(viii) when X and Z are nitrogen, then not all of R¹, R², and R³ are alkoxy; and

(ix) the compound is not a salt of any one (i)-(viii).

In another aspect, this invention is directed to a pharmaceutical composition comprising a compound of Formula (I) or a pharmaceutically acceptable salt thereof and a pharmaceutically acceptable excipient.

In still another aspect, this invention is directed to a method of treating a disorder treatable by inhibition of PDE10 enzyme in a patient which method comprises administering to the patient a pharmaceutical composition comprising a compound of Formula (I) or a pharmaceutically acceptable salt thereof and a pharmaceutically acceptable excipient. Preferably, the disease is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomer, mixtures of stereoisomers where the compound of Formula (I) has a stereochemical centre), a pharmaceutically acceptable salt thereof, or mixtures thereof.

### DETAILED DESCRIPTION

### Definitions

Unless otherwise stated, the following terms used in the specification and claims are defined for the purposes of this Application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated, e.g., methylene, ethylene, propylene, 1-methylpropylene, 2-methylpropylene, butylene, pentylene, and the like.

“Alkylthio” means a —SR radical, where R is alkyl as defined above, e.g., methylthio, ethylthio, and the like.

“Alkylsulfonyl” means a —SO₂R radical, where R is alkyl as defined above, e.g., methylsulfonyl, ethylsulfonyl, and the like.

“Amino” means an —NH₂.

“Alkylamino” means an —NHR radical, where R is alkyl as defined above, e.g., methylamino, ethylamino, propylamino, or 2-propylamino, and the like.

“Alkoxy” means an —OR radical, where R is alkyl as defined above, e.g., methoxy, ethoxy, propoxy, or 2-propoxy, n-, iso-, or tert-butoxy, and the like.

“Alkoxycarbonyl” means a —C(O)OR radical, where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means an —OR radical, where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two —NRR′, where R is hydrogen, alkyl, or —COR^{a}, where R^{a} is alkyl, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical, where R is aminoalkyl as defined above, e.g., 2-aminoethoxy, 2-dimethylaminopropoxy, and the like.

“Aminocarbonyl” means a —CONRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminocarbonyl, 2-dimethylaminocarbonyl, and the like.

“Aminosulfinyl” means a —SONRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminosulfinyl, 2-dimethylaminosulfinyl, and the like.

“Aminosulfonyl” means a —SO₂NRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetyl, propionyl, benzoyl, pyridinylcarbonyl, and the like. When R in a —COR radical is alkyl, the radical is also referred to herein as “alkylcarbonyl.”

“Acylamino” means an —NHCOR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetylamino, propionylamino, and the like.

“Aryl” means a monovalent monocyclic or bicyclic aromatic hydrocarbon radical of 6 to 12 ring atoms, e.g., phenyl or naphthyl.

“Aralkyl” means an -(alkylene)-R radical, where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl.

“Cycloalkylalkyl” means an -(alkylene)-R radical, where R is cycloalkyl as defined above; e.g., cyclopropylmethyl, cyclobutylmethyl, cyclopentylethyl, or cyclohexylmethyl, and the like.

“Cycloalkyloxy” means an —OR radical, where R is cycloalkyl as defined, e.g., cyclopropyloxy, cyclobutyloxy, cyclopentyloxy, cyclohexyloxy, and the like.

“Cycloalkylalkyloxy” means an —OR radical, where R is cycloalkylalkyl as defined, e.g., cyclopropylmethyloxy, cyclobutylmethyloxy, cyclopentylethyloxy, cyclohexylmethyloxy, and the like.

“Carboxy” means —COOH.

“Disubstituted amino” means an —NRR′ radical, where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., dimethylamino, phenylmethylamino, and the like.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means an —OR radical, where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that, if two hydroxy groups are present, they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical, where R is hydroxyalkyl as defined above.

“Heterocyclyl” means a saturated or unsaturated monovalent monocyclic group of 4 to 8 ring atoms in which one or two ring atoms are heteroatom independently selected from N, O, and S(O)ₙ, where n is an integer from 0 to 2, the remaining ring atoms being C. Additionally, one or two ring carbon atoms can optionally be replaced by a —CO— group and the heterocyclic ring may be fused to phenyl or heteroaryl ring, provided that the heterocyclyl ring is not phthalazin-1(2H)-one. Unless stated otherwise, the fused heterocyclyl ring can be attached at any ring atom. More specifically, the term heterocyclyl includes, but is not limited to, pyrrolidino, piperidino, 2-oxopyrrolidinyl, 2-oxopiperidinyl, homopiperidino, morpholino, piperazino, tetrahydropyranyl, thiomorpholino, and the like. When the heterocyclyl ring has five, six or seven ring atoms, and is not fused to phenyl or heteroaryl ring, it is referred to herein as “monocyclic five-, six-, or seven-membered heterocyclyl ring or five-, six-, or seven-membered heterocyclyl ring.” When the heterocyclyl ring is unsaturated it can contain one or two ring double bonds, provided that the ring is not aromatic.

“Heterocyclylalkyl” means an -(alkylene)-R radical, where R is heterocyclyl ring as defined above, e.g., tetrahydrofuranylmethyl, piperazinylmethyl, morpholinylethyl, and the like.

“Heteroaryl” means a monovalent monocyclic or bicyclic aromatic radical of 5 to 10 ring atoms, where one or more, preferably one, two, or three, ring atoms are heteroatoms independently selected from N, O, and S, and the remaining ring atoms are carbon, e.g., benzofuranyl, thiophenyl, imidazolyl, oxazolyl, quinolinyl, furanyl, thazolyl, pyridinyl, and the like.

“Heteroaralkyl” means an -(alkylene)-R radical, where R is heteroaryl as defined above.

“Monosubstituted amino” means an —NHR radical, where R is alkyl, acyl, sulfonyl, aryl, aralkyl, heteroaryl, heteroaralkyl, heterocyclyl, heterocyclylalkyl, cycloalkyl, cycloalkylalkyl, hydroxyalkyl, alkoxyalkyl, or aminoalkyl, each as defined above, e.g., methylamino, 2-phenylamino, hydroxyethylamino, and the like.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups by routine manipulation or in vivo. Prodrugs of compounds of Formula (I) include compounds wherein a hydroxy, amino, carboxylic, or a similar group is modified. Examples of prodrugs include, but are not limited to, esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol, or any group containing a nitrogen atom, these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for instance, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “pharmaceutically acceptable salt” also refers to salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are non-toxic. Additional information on suitable pharmaceutically acceptable salts can be found in Remington's Pharmaceutical Sciences, Gennaro, A. R. (Mack Publishing Company, 18th ed., 1995), which is incorporated herein by reference.

The compounds of the present invention may have one or more asymmetric centers. Compounds of the present invention containing an asymmetrically substituted atom may be isolated in an optically active, racemic, or diastereomeric form. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof, are within the scope of this invention.

Additionally, as used herein, the term “alkyl” includes all the possible isomeric forms of said alkyl group albeit only a few examples are set forth. Furthermore, when a cyclic group, such as aryl, heteroaryl, and heterocyclyl, is substituted, it includes all the positional isomers albeit only a few examples are set forth.

All polymorphic forms and hydrates of a compound of Formula (I) are also within the scope of this invention.

“Oxo” means the ═(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents, each independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, and sulfinyl, each as defined herein.

“Optionally substituted heteroaryl” means a monovalent monocyclic or bicyclic aromatic radical of 5 to 10 ring atoms, where one or more, preferably one, two, or three ring atoms are heteroatoms, each independently selected from N, O, and S, and the remaining ring atoms are carbon that is optionally substituted with one, two, or three substituents, each independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, and sulfinyl, each as defined herein. More specifically, the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl.

“Optionally substituted heterocyclyl” means a saturated or unsaturated monovalent cyclic group of 3 to 8 ring atoms in which one or two ring atoms are heteroatoms, each independently selected from N, O, and S(O)ₙ, where n is an integer from 0 to 2, and the remaining ring atoms are carbon. One or two ring carbon atoms can optionally be replaced by a —CO-(carbonyl) group and is optionally substituted with one, two, or three substituents, each independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, and sulfinyl, each as defined herein.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical, where R is alkyl, haloalkyl, aryl, aralkyl, heteroaryl, heteroaralkyl, heterocyclyl, heterocyclylalkyl, as defined above, e.g., methylsulfinyl, phenylsulfinyl, benzylsulfinyl, and the like.

“Sulfonyl” means a —SO₂R radical, where R is alkyl, haloalkyl, aryl, aralkyl, heteroaryl, heteroaralkyl, heterocyclyl, heterocyclylalkyl, as defined above, e.g., methylsulfonyl, phenylsulfonyl, benzylsulfonyl, pyridinylsulfonyl, and the like.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity, and the age, weight, etc., of the mammal to be treated.

### EMBODIMENTS

In one aspect, provided herein is a compound of Formula (I), an individual stereoisomer, a mixture of stereoisomers, or pharmaceutically acceptable salt thereof, as defined in the Summary of the Invention.

(1) In one embodiment, this invention is directed to compounds of Formula (I), wherein X and Y are nitrogen and Z is ═CH—.

(2) In another embodiment, this invention is directed to compounds of Formula (I), wherein Y and Z are nitrogen and X is —CH═.

(3) In yet another embodiment, this invention is directed to compounds of Formula (I), wherein X and Z are nitrogen and Y is ═CH—.

(4) In yet another embodiment, this invention is directed to compounds of Formula (I), wherein Y and Z are nitrogen and X is —CR=where R is alkyl.

(5) In another embodiment, this invention is directed to compounds of Formula (I), wherein Y and Z are nitrogen and X is —CR=where R is methyl, ethyl, n- or iso-propyl.

(6) In another embodiment, this invention is directed to compounds of Formula (I), wherein Y and Z are nitrogen and X is —CR=where R is halo. Within this embodiment, one group of compounds of Formula (I) is that wherein R is fluoro or chloro.

(7) In another embodiment, this invention is directed to compounds of Formula (I), wherein R¹ is hydrogen, R² is alkoxy, alkylamino, dialkylamino, fluoro, or trifluoromethyl, and R³ is selected from alkyl, alkoxy, cyano, halo, haloalkyl, haloalkoxy, and cycloalkyl; provided that, when X and Y or X and Z are nitrogen, and R¹ is hydrogen, then R² and R³ are not independently hydroxy, alkoxy, or haloalkoxy. Within this embodiment, one group of compounds is that wherein R³ is alkoxy, fluoro, or trifluoromethyl, and R² is alkyl.

(A) Within the above embodiments (1)-(7), and embodiments contained therein, one group of compounds of Formula (I) is that wherein R¹ is hydrogen.

(B) Within the above embodiment 2, and 4-6, another group of compounds of Formula (I) is that wherein R¹ is hydrogen and R² and R³ are alkoxy. In one class of compounds in this embodiment, R² is methoxy and R³ is methoxy, ethoxy, or propoxy.

(C) Within the above embodiments (1)-(7), another group of compounds of Formula (I) is that wherein R¹ is hydrogen, R² is alkoxy, and R³ is alkyl. Within this embodiment, one group of compounds of Formula (I) is that wherein R¹ is hydrogen, R² is methoxy, or ethoxy, and R³ is methyl, ethyl, or propyl.

(D) Within the above embodiments (1)-(7), one group of compounds of Formula (I) is that wherein R¹ is hydrogen, R² is alkoxy, and R³ is cycloalkyl, e.g., cyclopropyl. Within this embodiment, one group of compounds of Formula (I) is that wherein R¹ is hydrogen, R² is methoxy or ethoxy, and R³ is cyclopropyl.

(E) Within the above embodiments (1)-(7), one group of compounds of Formula (I) is that wherein R¹ is hydrogen, R² is fluoro, trifluoromethoxy, methylamino, or dimethylamino, and R³ is alkyl, alkoxy, haloalkyl, halo, or cycloalkyl.

(F) Within the above embodiments (1)-(7), one group of compounds of Formula (I) is that wherein R¹ is hydrogen, R³ is alkoxy, and R² is alkyl.

(G) Within the above embodiments (1)-(7), one group of compounds of Formula (I) is that wherein R¹ is hydrogen, R³ is alkoxy, and R² is cycloalkyl.

(i) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, one group of compounds of Formula (I) is that wherein R^{3a} is a ring of formula (a)

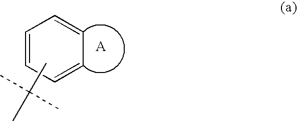

wherein A is a monocyclic five-, six-, or seven-membered heterocyclyl ring substituted with R⁴, R⁵ and R⁶ as defined in the Summary of the Invention.

(ii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), another group of compounds of Formula (I) is that wherein R^{3a} is a ring of formula (b):

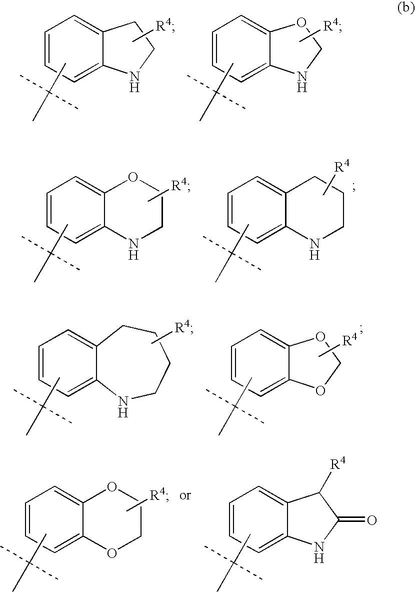

(iii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, another group of compounds of Formula (I) is that wherein R^{3a} is a ring of formula (c):

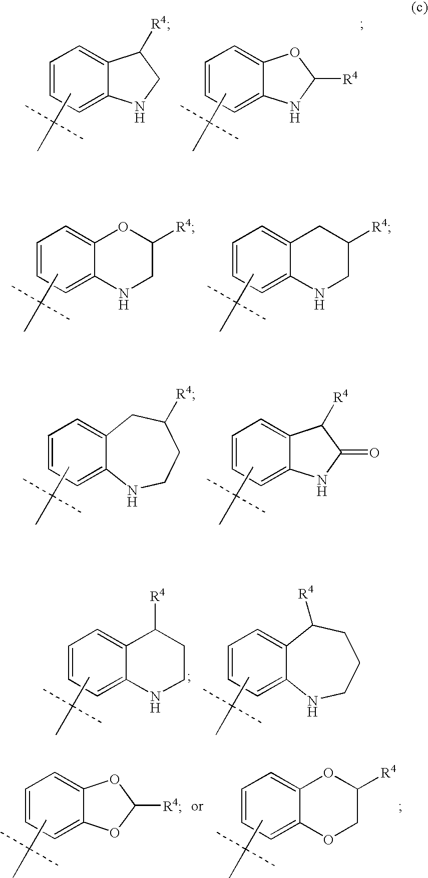

(iv) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (e):

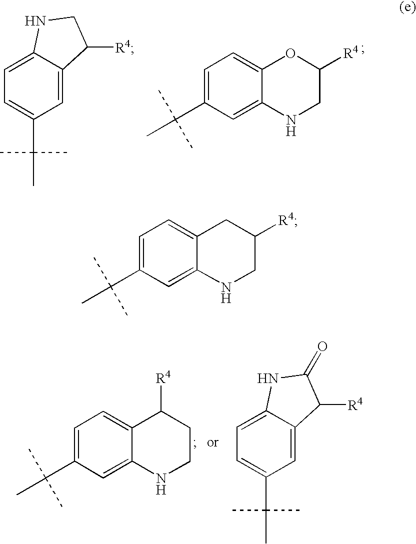

The R⁴ group in (ii)-(iv) is as defined in the Summary of the invention. Within the subgroups (ii)-(iv) above, one group of compounds is that wherein R⁴ is phenyl optionally substituted as defined in the Summary of the Invention.

Within the subgroups (ii)-(iv) above, another group of compounds is that wherein R⁴ is heteroaryl optionally substituted as defined in the Summary of the Invention.

Within the subgroups (ii)-(iv)) above, another group of compounds is that wherein R⁴ is a saturated monocyclic heterocyclyl optionally substituted as defined in the Summary of the Invention.

Within the subgroups (ii)-(iv) above, another group of compounds is that wherein R^{3a} is saturated fused heterocyclyl optionally substituted as defined in the Summary of the Invention.

The R^{3a} rings in subgroups (ii)-(iv) above, the subgroups contained therein, including the hydrogen in —NH— groups in the rings, can also be optionally substituted with R⁵ and R⁶ are as defined in the Summary of the Invention. Preferably, one of R⁵ and R⁶ is hydrogen.

(v) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (f):

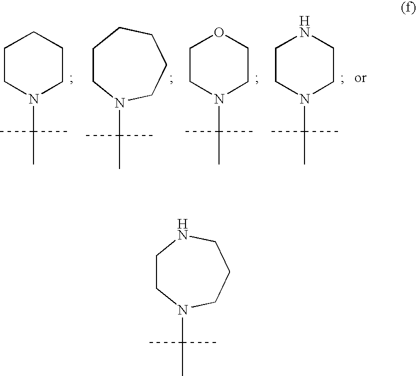

wherein the ring is substituted with R⁴, R⁵ and R⁶ as defined in the Summary of the Invention. Within this subgroup, one group of compounds is that wherein the above rings are substituted with R⁴ as defined in the Summary of the Invention and substituted with R⁵ and R⁶, where one of R⁵ and R⁶ is hydrogen. In one group of compounds, the —NH— group in the rings is substituted with alkyl, cycloalkyl, or cycloalkylalkyl. In another group of compounds, the —NH— group in the rings is unsubstituted. Within this embodiment, one group of compounds is that wherein R^{3a} is morpholin-1-yl or piperazin-1-yl substituted, as defined in (v) above. Within this embodiment, another group of compounds is that wherein R^{3a} is piperidin-1-yl or homopiperidin-1-yl, substituted as defined in (v) above.

(vi) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (g):

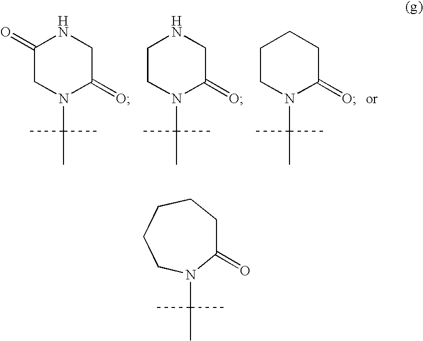

wherein the ring is substituted with R⁴, R⁵ and R⁶ as defined in the Summary of the Invention. Within this subgroup, one group of compounds is that wherein the above rings are substituted with R⁴ as defined in the Summary of the Invention, preferably, aryl, heteroaryl, or six-membered saturated heterocyclyl optionally substituted with R^{a}, R^{b} and R^{c} and substituted with R⁵ and R⁶, where one of R⁵ and R⁶ is hydrogen. In one group of compounds, the —NH— group in the rings is substituted with alkyl, cycloalkyl, or cycloalkylalkyl. In another group of compounds, the —NH— group in the rings is unsubstituted.

(vii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (h):

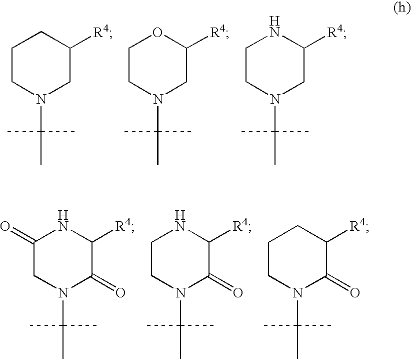

wherein R⁴ is as defined in the Summary of the Invention. Within this embodiment, one group of compounds is that wherein R⁴ is cycloalkyl, phenyl, heteroaryl, or six-membered saturated heterocyclyl optionally substituted with R^{a}, R^{b} and R^{c}; and the rings are optionally substituted, including the hydrogen atom on the —NH— group within the ring, with R⁵ and R⁶ as defined in the Summary of the Invention, preferably, R⁵ is hydrogen and R⁶ is attached to the carbon adjacent to the nitrogen attached to the cinnoline, quinazoline, or phthalazine ring.

(viii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (I):

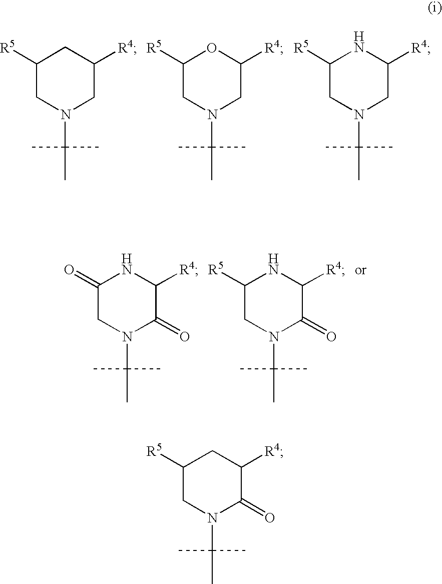

wherein:

R⁴ is phenyl or heteroaryl, substituted at the para position with R^{a} and optionally substituted with R^{b} and R^{c}, wherein R^{a}, R^{b}, R^{c}, and R⁵ are as defined in the Summary of the Invention. The —NH— group in the above rings can optionally be substituted with R⁶, as defined in the Summary of the Invention. In one group of compounds within this embodiment, R⁶ is cycloalkyl, alkyl, or cycloalkylalkyl. In one group of compounds within this embodiment, R^{3a} is morpholin-4-yl or piperazin-1-yl, where R⁴ is phenyl substituted with R^{a} and R^{b}, which are meta to each other. In another group of compounds within this embodiment, R^{3a} is piperidin-1-yl substituted as described above. In yet another group of compounds within this embodiment, R⁴ is —NHCOR⁷, where R⁷ is aryl or heteroaryl, as defined in the Summary of the Invention.

(ix) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a ring of formula 0):

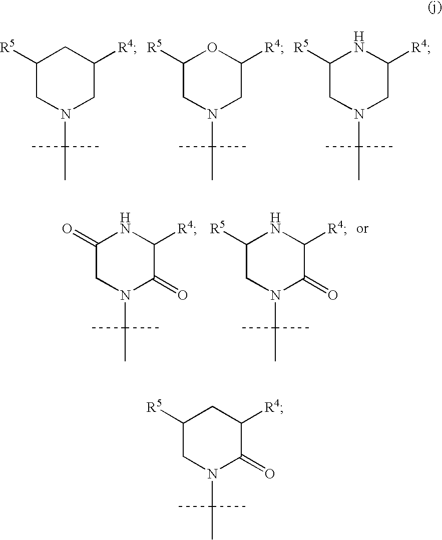

wherein:

R⁴ is heterocyclyl, preferably heterocyclyl containing at least a —C═O group, wherein the heterocyclyl ring is optionally substituted at the para position with R^{a}, and optionally substituted with R^{b} and R^{c}, wherein R^{a}, R^{b}, R^{c} and R⁵ are as defined in the Summary of the Invention. Within this group, in one embodiment, R⁴ is monocyclic saturated six-membered ring containing at least a —C═O group, optionally substituted at the para position with R^{a}, and optionally substituted with R^{b} and R^{c}, wherein R^{a}, R^{b}, and R^{c} are as defined in the Summary of the Invention. The —NH— group in the above rings can optionally be substituted with R⁶ as defined in the Summary of the Invention. Preferably, R⁶ is cycloalkyl, alkyl, or cycloalkylalkyl. In one group of compounds within this embodiment R^{3a} is other than piperidin-1-yl substituted as described above. In another group of compounds within this embodiment, R^{3a} is piperidin-1-yl substituted as described above.

(x) Within the above embodiments (1)-(7), and groups or embodiments contained, therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a ring of formula (k):

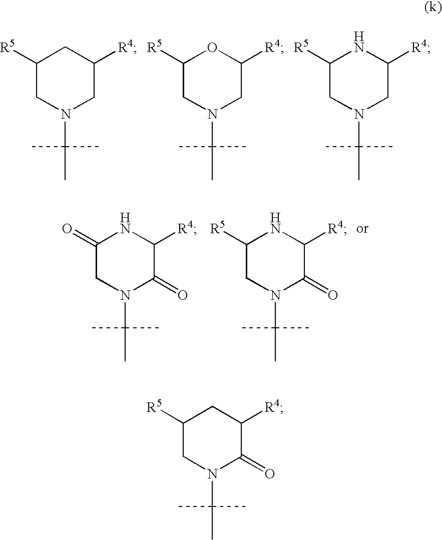

wherein:

R⁴ is cycloalkyl substituted at the para position with R^{a} and optionally substituted with R^{b} and R^{c}, wherein R^{a}, R^{b}, R^{c}, and R⁵ are as defined in the Summary of the Invention. The —NH— group in the above rings can optionally be substituted with R⁶ as defined in the Summary of the Invention. In one group of compounds within this embodiment, R⁶ is cycloalkyl, alkyl, or cycloalkylalkyl.

(xi) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (I):

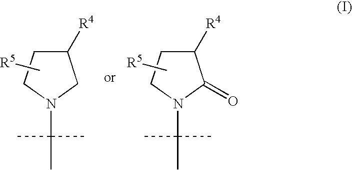

wherein R⁴ and R⁵ are as defined in the Summary of the Invention.

(xii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (b). In one group of compounds is that wherein R^{3a} is a ring of formula (m):

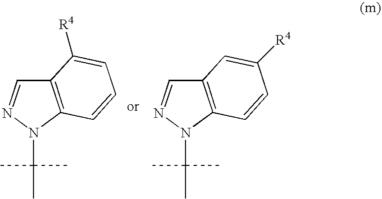

wherein:

R⁴ is cycloalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); more preferably phenyl, heteroaryl, or heterocyclyl; and optionally substituted with R⁵ and R⁶, which are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; and

wherein the aromatic or alicyclic ring in R⁴, R⁵, R⁶, and R⁷ is optionally substituted with one to three substitutents independently selected from R^{a}, R^{b}, and R^{c}, which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; and additionally substituted with one or two substitutents independently selected from R^{d} and R^{e}, where R^{d} and R^{e} are chloro or fluoro.

Within this embodiment, one group of compounds is that wherein R^{3a} is:

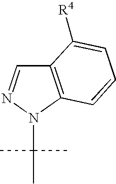

wherein R⁴ is phenyl, heteroaryl, or five- or six-membered heterocyclyl, optionally substituted with one to three substitutents independently selected from R^{a}, R^{b}, and R^{c}, as defined in the Summary of the Invention.

Within this embodiment, another group of compounds is that wherein R^{3a} is:

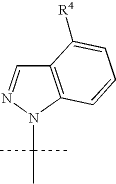

wherein R⁴ is morpholin-4-yl, piperazin-1-yl, or pyridinyl, optionally substituted with one to three substitutents independently selected from R^{a}, R^{b}, and R^{c}, as defined in the Summary of the Invention.

(xiii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (n):

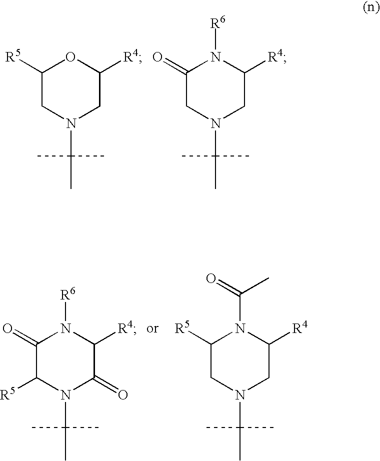

wherein:

R⁴ is cyclopentyl, cyclohexyl, phenyl, heteroaryl, or monocyclic saturated five- or six-membered heterocyclyl ring; R⁵ is hydrogen, alkyl, phenyl, heteroaryl, or monocyclic five- or six-membered heterocyclyl ring; and R⁶ is alkyl, preferably methyl; and wherein the aromatic or alicyclic ring in R⁴ and R⁵ are optionally substituted with R^{a}, R^{b} and R^{c}, as defined in the Summary of the Invention. Within this subgroup, in one embodiment, R⁴ is phenyl, heteroaryl, or monocyclic five- or six-membered heterocyclyl ring, and R⁵ is hydrogen or alkyl. In another embodiment, R⁴ and R⁵ are independently phenyl, heteroaryl, or monocyclic saturated five- or six-membered heterocyclyl ring. In each of the above embodiments, the aromatic or alicyclic ring are optionally substituted with R^{a} selected from alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and R^{b} and R^{c} independently selected from alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino.

(xiv) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (o):

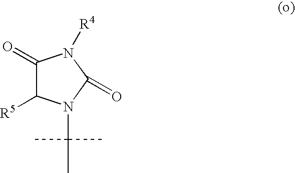

where R⁴ is aralkyl, preferably benzyl optionally substituted with R^{a}, R^{b}, R^{c}, and R⁵, as defined in the Summary of the Invention, preferably, R⁵ is hydrogen or alkyl.

(xv) Within the above (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (a):

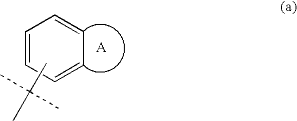

wherein A is a monocyclic five-, six-, or seven-membered heterocyclyl ring; and the ring (a) is substituted with:

R⁴, where R⁴ is cycloalkyl; cycloalkylalkyl; aryl; heteroaryl; heterocyclyl; aralkyl; heteroaralkyl; heterocyclylalkyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, or disubstituted amino; preferably hydrogen;

wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; or additionally substituted with one or two substitutents independently selected from R^{d} and R^{e}, where R^{d} and R^{e} are chloro or fluoro. In one embodiment, A is a saturated five- or six-membered heterocyclyl ring and substituted as described above.

(xvi) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a ring of formula (b):

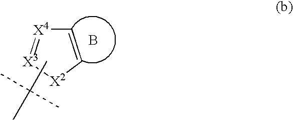

where:

X², X³, and X⁴ are independently carbon, nitrogen, oxygen, or sulfur; provided that at least two of X², X³, and X⁴ are other than carbon; and

B is phenyl; a six-membered heteroaryl ring (wherein the six-membered heteroaryl ring contains one or two nitrogen atoms, the rest of the ring atoms being carbon); or a monocyclic five-, six-, or seven-membered heterocyclyl ring; and wherein ring (b) is substituted with:

R⁴, where R⁴ is cycloalkyl; cycloalkylalkyl; aryl; heteroaryl; heterocyclyl; aralkyl; heteroaralkyl; heterocyclylalkyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; preferably hydrogen; and

wherein the aromatic or alicyclic ring in R⁴, R⁵, R⁶, and R⁷ is optionally substituted with one to three substitutents independently selected from R^{a}, R^{b}, and R^{c}, which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e}, where R^{d} and R^{e} are chloro or fluoro.

(xvii) Within the above embodiments (1)-(7), and embodiments contained therein, i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a monocyclic six- or seven-membered heterocyclyl ring, substituted with:

R⁴, where R⁴ is selected from aryl; heteroaryl; heterocyclyl; aralkyl; heteroaralkyl; heterocyclylalkyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and

R⁵, where R⁵ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; preferably hydrogen; and

wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

In one group within this embodiment, R^{3a} is other than piperidinyl substituted as described above. In one group within this embodiment, R^{3a} is piperidinyl substituted as described above.

(xviii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is pyrrolidin-1-yl substituted with:

R⁴, where R⁴ is cycloalkyl; cycloalkylalkyl; aryl; heteroaryl; heterocyclyl; aralkyl; heteroaralkyl; heterocyclylalkyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; preferably hydrogen; and

wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e}, where R^{d} and R^{e} are chloro or fluoro.

(xix) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is 2-oxopyrrolidinyl or 2,4-dioxoimidazolidinyl substituted with:

R⁴, where R⁴ is cycloalkyl; cycloalkylalkyl; aryl; heteroaryl; heterocyclyl; aralkyl; heteroaralkyl; heterocyclylalkyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; preferably hydrogen; and

wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e}, where R^{d} and R^{e} are chloro or fluoro.

(xx) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is phenyl optionally substituted as defined in the Summary of the Invention. Within this embodiment, one group of compounds is that wherein R^{3a} is a group of formula:

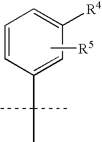

wherein one of R⁴ and R⁵ is hydrogen; alkyl; halo; haloalkyl; alkoxy; haloalkoxy; cyano; amino; monsubstituted or disubstituted amino; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and the other of R⁴ and R⁵ is cycloalkyl, aryl, heteroaryl, or heterocyclyl; and

wherein the aromatic or alicyclic ring in R⁴ and R⁵ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is aryl, heteroaryl, or heterocyclyl optionally substituted with one to three substitutents independently selected from R^{a}, R^{b}, and R^{c}.

(xxi) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

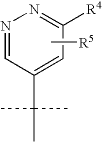

wherein R⁴ and R⁵ are as defined in (xvii) above.

(xxii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

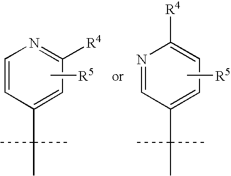

wherein R⁴ and R⁵ are as defined in (xxi) above. Within this subgroup (xxii), another class of compounds is that wherein R⁴ is heteroaryl optionally substituted with one to three substitutents independently selected from R^{a}, R^{b}, and R^{c}. Within this subgroup (xxi), another class of compounds is that wherein R⁴ is heterocyclyl, preferably piperazinyl, piperidinyl, or morpholinyl, optionally substituted with one to three substitutents independently selected from R^{a}, R^{b}, and R^{c}. Within this subgroup (xxi), another class of compounds is that where R⁴ is monosubstituted or disubstituted amino, and R⁵ is hydrogen, alkyl, or halo.

(xxiii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

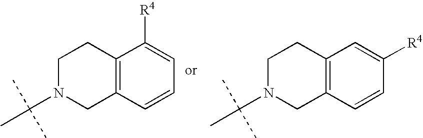

wherein R⁴ is as defined in the Summary of the Invention. The isoquinoline ring can optionally be substituted with R⁵ as defined in the Summary of the Invention. Within this subgroup (xxiii), another class of compounds is that where R⁴ is heteroaryl, optionally substituted with one to three substitutents independently selected from R^{a}, R^{b}, and R^{c}. Within this subgroup (xxiii), another class of compounds is that where R⁴ is heterocyclyl, preferably piperazinyl, piperidinyl, or morpholinyl, optionally substituted with one to three substitutents independently selected from R^{a}, R^{b}, and R^{c}.

(xxiv) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

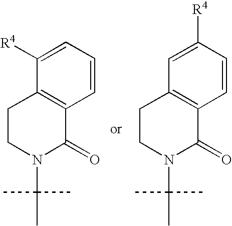

wherein R⁴ is as defined in the Summary of the Invention. The isoquinoline ring can optionally be substituted with R⁵ as defined in the Summary of the Invention. Within this subgroup (xxiv), another class of compounds is that wherein R⁴ is heteroaryl optionally substituted with one to three substitutents independently selected from R^{a}, R^{b}, and R^{c}. Within this subgroup (xxiv), another class of compounds is that wherein R⁴ is heterocyclyl, preferably piperazinyl, piperidinyl, or morpholinyl, optionally substituted with one to three substitutents independently selected from R^{a}, R^{b}, and R^{c}.

(xxv) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

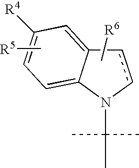

wherein R⁴, R⁵, and R⁶ are as defined in the Summary of the Invention. Within this embodiment, one class of compounds is that wherein R^{3a} is a group of formula:

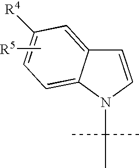

wherein one of R⁴ and R⁵ is hydrogen, alkyl, halo, haloalkyl, alkoxy, haloalkoxy, cyano, amino, monsubstituted or disubstituted amino, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and the other one of R⁴ and R⁵ is aryl, heteroaryl, or heterocyclyl; and
wherein the aromatic or alicyclic ring in R⁴ and R⁵ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is aryl, heteroaryl, or heterocyclyl optionally substituted with one to three substitutents independently selected from R^{a}, R^{b}, and R^{c}.

Within this embodiment, another class of compounds is that of formula:

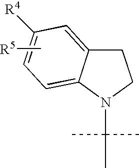

wherein R⁴ and R⁵ are as described immediately above.

(xxvi) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, another class of compounds is that wherein R^{3a} is a group of formula:

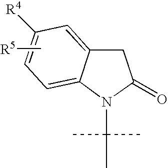

wherein R⁴ and R⁵ are as described immediately above.

(xxvii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

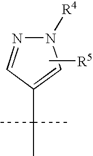

wherein R⁵ is hydrogen or alkyl and R⁴ is aryl, heteroaryl, aralkyl, heteroaralkyl, or heterocyclyl optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. In one embodiment, R⁴ is aralkyl, preferably benzyl, optionally substituted with one to three substitutents independently selected from R^{a}, R^{b}, and R^{c}. In another embodiment, R⁴ is heteroaryl, optionally substituted with one to three substitutents independently selected from R^{a}, R^{b}, and R^{c}. In another embodiment, R⁴ is heterocyclyl, optionally substituted with optionally substituted phenyl, optionally substituted heteroaryl. Preferably, R^{3a} is a group of formula:

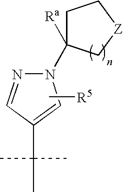

wherein R⁵ is hydrogen or alkyl, preferably hydrogen; n is 1, 2, or 3; Z is —O—, —NH—, or —N-(alkylene)-; and R^{a} is phenyl or heteroaryl, optionally substituted with R^{a}, R^{b}, and R^{c}, preferably phenyl optionally substituted with R^{a}, R^{b}, and R^{c}.

(xxviii) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, and groups contained therein, yet another group of compounds of Formula (I) is that wherein R^{3a} is a group of formula:

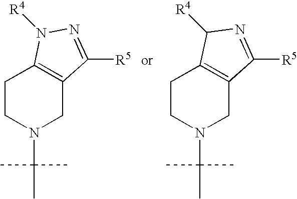

wherein one of R⁴ and R⁵ is hydrogen; alkyl; halo; haloalkyl; alkoxy; haloalkoxy; cyano; amino; monsubstituted or disubstituted amino; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and the other of R⁴ and R⁵ is aryl, heteroaryl, or heterocyclyl; and
wherein the aromatic or alicyclic ring in R⁴ and R⁵ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

Within this embodiment, one group of compounds is that wherein R⁴ is phenyl, heteroaryl, or heterocyclyl optionally substituted with one to three substitutents independently selected from R^{a}, R^{b}, and R^{c}.

(xxix) Within the above embodiments (1)-(7), and groups or embodiments contained therein, i.e., (1)(A, C-G), (2)(A-G), (3)(A, C-G), (4)(A-G), (5)(A-G), (6)(A-G), 7(A, C-G), and groups contained therein, yet another group of compounds of Formula (I) is that wherein R^{3a} is a group of formula:

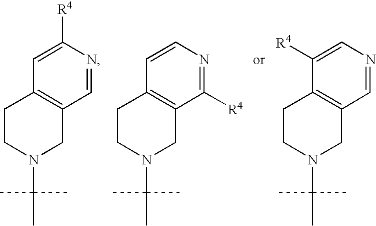

wherein R⁴ is alkyl; haloalkoxy; cycloalkyl; aryl; heteroaryl; heterocyclyl; or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and

wherein the aromatic or alicyclic ring in R⁴ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is cycloalkyl, aryl, heteroaryl, or heterocyclyl optionally substituted with one to three substitutents independently selected from R^{a}, R^{b}, and R^{c}.

(xxx) Within the above embodiments (1)-(7), and embodiments contained therein i.e., (1)(A, C-G), (2)(A-G), (3)(A, C-G), (4)(A-G), (5)(A-G), (6)(A-G), 7(A, C-G), and groups or embodiments contained therein, yet another group of compounds of Formula (I) is that wherein R^{3a} is a group of formula:

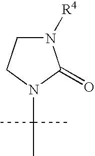

wherein R⁴ is aralkyl, preferably benzyl optionally substituted with R^{a}, R^{b} and R^{c} as defined in the Summary of the Invention.

Representative compounds of Formula (I) are provided in Table I below:

TABLE 1

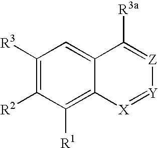

| Cpd. | | | | | | | |
|---|---|---|---|---|---|---|---|
| # | X | Y | Z | R¹ | R² | R³ | R^{3a} |
| 1 | N | N | CH | H | methoxy | ethyl | 7-methoxy-3,4-dihydro-isoquinolin- |
| | | | | | | | 1(2H)-one-1-yl formate salt |
| 2 | N | N | CH | H | ethyl | methoxy | 6,7-dimethoxy-1,2,3,4-tetrahydro- |
| | | | | | | | isoquinolin-2-yl formate salt |
| 3 | N | N | CH | H | ethyl | methoxy | 7-methoxy-3,4-dihydro-isoquinolin- |
| | | | | | | | 1(2H)-one-1-yl formate salt |
| 4 | N | N | CH | H | ethyl | methoxy | 3-cyclopropylamino-carbonyl-4,5,6,7- |
| | | | | | | | tetrahydro-1H-pyrazolo[4,3-c]pyridin-5- |
| | | | | | | | yl |
| 5 | N | N | CH | H | ethyl | methoxy | 1-benzylpyrazol-4-yl formate salt |
| 6 | N | N | CH | H | ethyl | methoxy | 3-(2-oxopiperidin-2-yl)-piperidin-1-yl |
| 7 | N | N | CH | H | methoxy | ethyl | 1-benzylpyrazol-4-yl |
| 8 | N | N | CH | H | methoxy | ethyl | 2-(4-methoxyphenyl)-morpholin-4-yl |
| 9 | N | N | CH | H | fluoro | methoxy | 2-(4-methoxyphenyl)-morpholin-4-yl |
| 10 | CH | N | N | H | methoxy | methoxy | 3-(2-oxopiperidin-2-yl)piperidin-1-yl |
| 11 | N | N | CH | H | methoxy | ethyl | 3-(2-oxopiperidin-2-yl)piperidin-1-yl |
| 12 | N | N | CH | H | fluoro | methoxy | 3-(2-oxopiperidin-2-yl)piperidin-1-yl |
| 13 | CH | N | N | H | methoxy | methoxy | 2-(4-methoxyphenyl)-morpholin-4-yl |
| 14 | N | N | CH | H | fluoro | methoxy | 1-benzylpyrazol-4-yl |
| 15 | N | N | CH | H | methyl | methoxy | 2-(4-methoxyphenyl)-morpholin-4-yl |
| 16 | N | N | CH | methoxy | methyl | H | 2-(4-methoxyphenyl)-morpholin-4-yl |
| 17 | N | N | CH | H | methoxy | CH₃ | 3-(2-oxopiperidin-2-yl)-piperidin-1-yl |
| 18 | N | N | CH | OCH₃ | methyl | H | 3-(2-oxopiperidin-2-yl)-piperidin-1-yl |
| 19 | N | N | CH | H | methoxy | methyl | 2-(morpholin-4-yl)pyridin-5-yl |
| 20 | N | N | CH | H | methyl | methoxy | 2-(4-methoxyphenyl)-morpholin-4-yl |
| 21 | N | N | CH | H | fluoro | methoxy | 3S-(2-oxopiperidin-2-yl)piperidin-1-yl |
| 22 | N | N | CH | H | fluoro | methoxy | 3R-(2-oxopiperidin-2-yl)-piperidin-1-yl |
| 23 | N | N | CH | H | methyl | methoxy | 3-(2-oxopiperidin-2-yl)-piperidin-1-yl |
| 24 | N | N | CH | H | H | methoxy | 2-(4-methoxyphenyl)-morpholin-4-yl |
| 25 | N | N | CH | H | methyl | methoxy | 2R-(4-methoxyphenyl)-morpholin-4-yl |
| 26 | N | N | CH | H | methyl | methoxy | 2S-(4-methoxyphenyl)-morpholin-4-yl |
| 27 | N | N | CH | H | methyl | ethoxy | 2-(4-methoxyphenyl)-morpholin-4-yl |
| 28 | N | N | CH | H | chloro | methoxy | 2-morpholin-4-ylpyridin-5-yl |
| 29 | N | N | CH | H | fluoro | methoxy | 2-morpholin-4-ylpyridin-5-yl |
| 30 | N | N | CH | methoxy | methoxy | methoxy | 2-(3,5-dimethoxyphenyl)piperazine- |
| | | | | | | | 1-yl |

### General Synthetic Schemes

Compounds of this invention can be made by the methods depicted in the reaction schemes shown below.

The starting materials and reagents used in preparing these compounds are either available from commercial suppliers such as Aldrich Chemical Co. (Milwaukee, Wis.), Bachem (Torrance, Calif.), or Sigma (St. Louis, Mo.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including, but not limited to, filtration, distillation, crystallization, chromatography, and the like. Such materials may be characterized using conventional means, including physical constants and spectral data.

Unless specified to the contrary, the reactions described herein take place at atmospheric pressure over a temperature range from about −78° C. to about 150° C., from about 0° C. to about 125° C., or from at about room (or ambient) temperature, e.g., about 23° C.

Compounds of Formula (I), where X and Y are nitrogen, Z is carbon, and R¹, R², R³, and R^{3a} are as defined in the Summary of the Invention, can be prepared as described in Scheme 1 below.

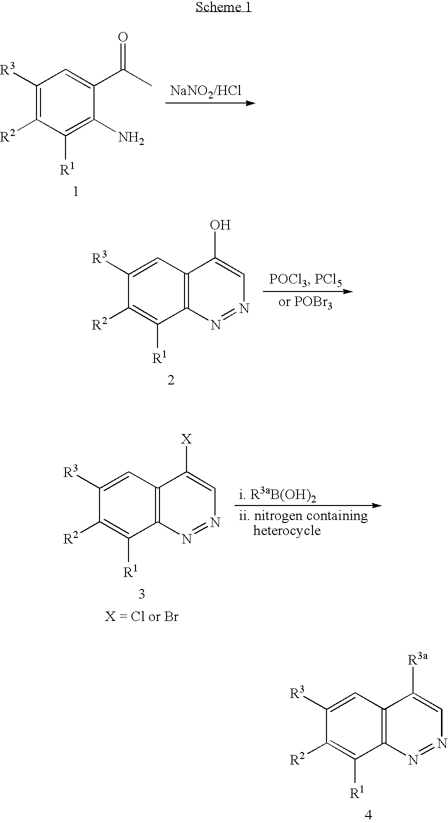

Treatment of 2-aminoacetophenone 1 with sodium nitrite in concentrated hydrochloric acid and water provides a diazo compound intermediate that cyclizes upon heating to provide 4-hydroxycinnoline 2. Treatment of compound 2 with either phosphorous oxychloride or phosphorous oxybromide provides the corresponding chloro or bromo compound 3. The chloro derivative 3 is prepared by heating 2 in neat phosphorous oxychloride, followed by recrystallization of the product after neutralization (see, Castle et al., J. Org. Chem. 17:1571, 1952). The bromo derivative 3 is prepared by mixing a concentrated suspension of 4-hydroxycinnoline 2 in chloroform and phosphorous oxybromide at room temperature, and then warming to reflux for 8 to 16 h. Extractive workup after neutralization and subsequent recrystallization from alcoholic solvent such as ethanol provides 4-bromocinnoline.

In addition to chloro and bromo, other leaving groups, such as triflate, mesylate, tosylate, and the like, are also suitable as X in cinnoline derivative 3. These derivatives can be readily prepared by reacting 4-hydroxycinnoline 2 with trifluoromethansulfonyl chloride, mesyl chloride, and tosyl chloride, respectively, under conditions well known in the art.

2-Aminoacetophenone derivatives 1 are either commercially available or can be readily synthesized by methods well known in the art. For example, a 2-aminoacetophenone derivative 1, wherein R³ is alkyl and R² is alkoxy, is prepared according to Scheme 2, which exemplifies the synthesis of 1-(2-amino-5-ethyl-4-methoxy phenyl)ethanone.

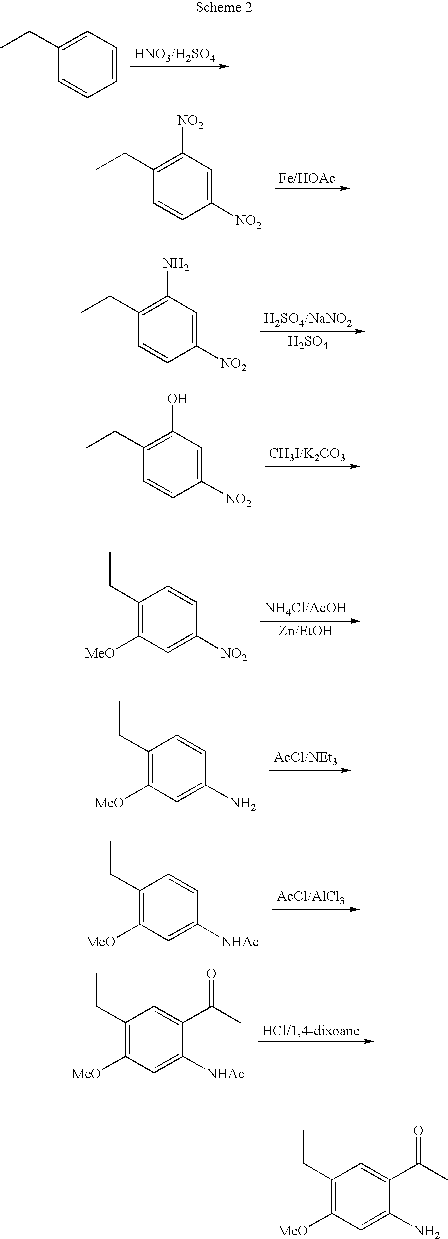

2-Aminoacetophenone 1, wherein R³ is alkoxy and R² is alkyl, is prepared as shown in Scheme 3, which exemplifies the synthesis of 1-(2-amino-4-ethyl-5-methoxyphenyl)ethanone.

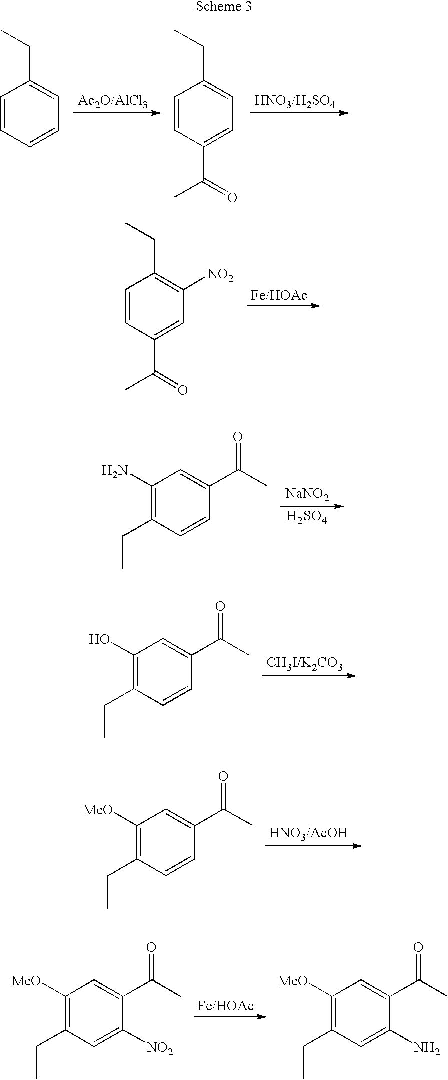

Cinnoline derivative 3 in Scheme 1 is then converted to the corresponding compound of Formula (I) via a variety of synthetic methods known to one of ordinary skill in the art. For example, compounds of Formula (I), wherein R^{3a} is an aryl or heteroaryl ring, are by Suzuki-type coupling of the corresponding aryl or heteroaryl boronic acid with compound 3 where X is halo (see, e.g., Miyaura and Suzuki, Chem. Rev. 95:2457-2483, 1995). Such boronic acids are either commercially available (e.g., Aldrich Chemical Co. (Milwaukee, Wis.), Lancaster Synthesis (Ward Hill, Mass.), or Maybridge (Conrwall, UK)) or can readily be prepared from the corresponding bromides by methods described in the literature (see, e.g., Miyaura et al., Tetrahedron Letters 1979, 3437; Miyaura and Suzuki, Chem. Commun. 1979, 866).

Compounds of Formula (I), where R^{3a} is a heterocyclic ring (e.g., pyrrolidin-1-yl, piperidin-1-yl, morpolin-4-yl) attached via a nitrogen atom and the like, are prepared by reacting cinnoline derivative 3, where X is halo or other suitable leaving group such as tosylate, triflate, mesylate and the like, with the heterocyclic ring in the presence of a base such as triethylamine and pyridine. Suitable solvents include, but are not limited to, tetrahydrofuran (THF) and DMF. Such heterocyclic rings (pyrrolidines, piperidines, homopiperidines, piperazines, homopiperazines, morpholines, and the like) are either commercially available or can be readily prepared by standard methods known within the art (see, e.g., Louie and Hartwig, Tetrahedron Letters 36:3609, 1995; Guram et al., Angew Chem. Int. Ed. 34:1348, 1995). Alternatively, a compound of Formula (I) is prepared by heating 3 with the heterocyclic ring in a suitable organic solvent, such as THF, benzene, dioxane, toluene, alcohol, or a mixture thereof, under catalytic conditions using, for example, a palladium or copper catalyst, in the presence of a suitable base, such as potassium carbonate, sodium t-butoxide, lithium hexamethyldisilizane, and the like. Suitable catalysts include, but are not limited to, tris(dibenzylideneacetone) dipalladium(0) and copper (I) iodide)

Substituted indazoles useful to make compounds of Formula (I) are either commercially available (e.g., Aldrich Chemical Co., Sinova, Inc. (Bethesda, Mass.), J & W PharmLab, LLC (Morrisville, Pa.)) or can be prepared by methods commonly known within the art (see, e.g., Lebedev et al., J. Org. Chem. 70(2):596-602, 2005; and the references cited therein). For example, indazoles, wherein R⁴ is heterocyclyl (e.g., morpholine or N-methylpiperazine), may be synthesized by Buchwald-type coupling of the corresponding bromoindazole with the desired heterocyclic compound. The bromoindazoles may be prepared as described in International Publication No. WO 2004/029050. Copper catalyzed reaction of the appropriately substituted indazole with 3 (where X is halo) provides the appropriate compound of Formula (I). Alternatively, the bromoindazole undergoes palladium catalyzed reaction with compound 3 (X is halo) to provide a 4-(bromo-1H-indazol-1-yl) substituted compound of Formula (I). Subsequent N-arylation reaction with, for example, morpholine or N-methylpiperazine, provides the desired compound of Formula I. Furthermore, Suzuki-type reaction of the 4-(bromo-1H-indazol-1-yl)-substituted cinnoline compound with aryl or heteroaryl boronic acids (e.g., phenylboronic acid or 4-pyridine boronic acid) gives the corresponding 4-(aryl or heteroaryl substituted indazole)cinnoline compound of Formula (I).

Compounds of Formula (I), where X and Z are nitrogen, Y is carbon, and R¹, R², R³ and R^{3a} are as defined in the Summary of the Invention, can be prepared as described in Scheme 4 below.

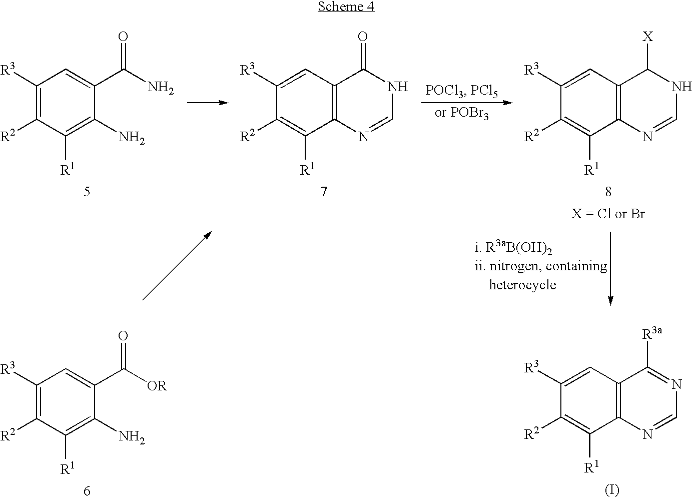

Reaction of 2-aminobenzamide 5 with trimethyl orthoformate or 2-aminobenzoic ester of compound 6 with formamide in the presence of a base, such as ammonium carbonate, provides the corresponding 4-hydroxyquinazolone 7, which, upon treatment with either phosphorous oxychloride or phosphorous oxybromide, provides the corresponding chloro or bromo derivative 8. The chloro derivative 8 is prepared by heating 7 in neat phosphorous oxychloride, followed by recrystallization of the product after neutralization (see, e.g. Castle et al., J. Org. Chem. 17:1571, 1952). The bromo derivative 8 is prepared by mixing a concentrated suspension of the 4-hydroxyquinazoline 7 in chloroform and phosphorous oxybromide at room temperature and then warming to reflux for 8 to 16 h. Extractive workup after neutralization and subsequent recrystallization from alcoholic solvent, such as ethanol, provides 4-bromoquinazoline 8. Compound 8 is then converted to a compound of Formula (I) as described in Scheme 1 above.

Compounds 5 and 6 are either commercially available or can be synthesized by methods common to the art.

Compounds of Formula (I), where Y and Z are nitrogen, X is CH, and R¹, R², R³ and R^{3a} are as defined in the Summary of the Invention, can be prepared as described in Scheme 5 below (Bioorg. Med. Chem. Lett. 10:2235, 2000).

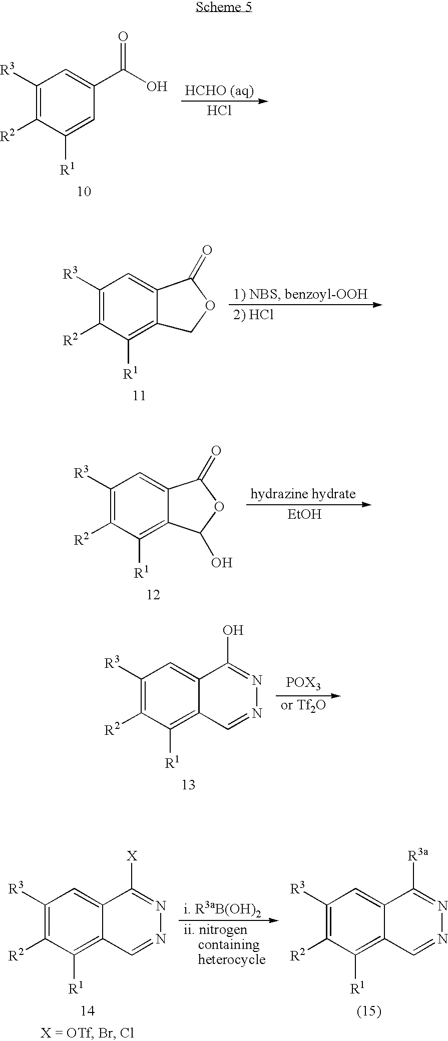

Treatment of compound 10 with aqueous formaldehyde and hydrochloric acid provides the cyclized ester 11. Compound 10 is either commercially available (e.g., 3,4-dimethoxy benzoic acid) or can be synthesized by methods common to the art (Bioorg. Med. Chem. Lett. 11:33, 2001). Oxidation of compound 11 with a suitable oxidizing agent, such as perbenzoic acid, in the presence of N-bromosuccinimide, followed by treatment with hydrazine, provides 4-hydroxy phthalazines 13. Treatment of 13 with phosphorous oxyhalide or with triflic anhydride, as described in Scheme 1 above, provides the 4-halo or triflyl phthalazines 14. Compound 14 is then converted to a compound of Formula (I) as described in Scheme 1 above.

Compounds of Formula (I), where Y and Z are nitrogen, X is CR where R is alkyl or halo, and R¹, R², R³ and R^{3a} are as defined in the Summary of the Invention, can be prepared as described in Scheme 6 below (J. Med. Chem. 39:343, 1996).

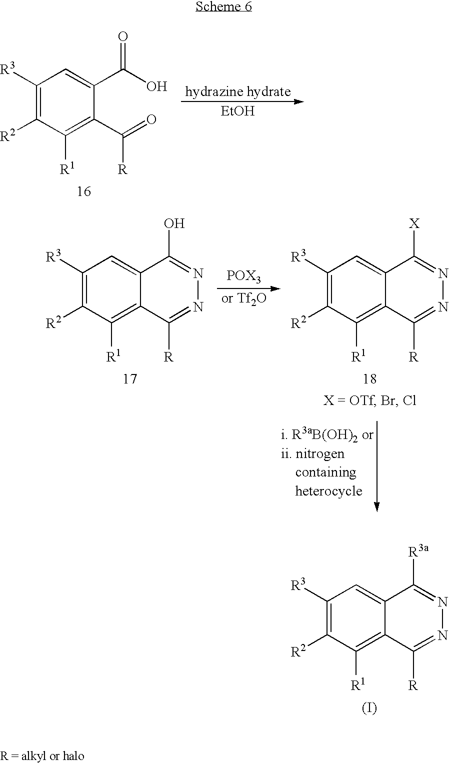

Compounds of Formula (I), where Y and Z are nitrogen, X is CR where R is alkyl or halo, and R¹, R², R³ and R^{3a} are as defined in the Summary of the Invention, are prepared by reacting 2-ketobenzoic acid (R is alkyl) or 2-carboxy acid halide (R is halo) 16 with hydrazine hydrate to provide 4-hydroxyphthalazine 17. Compound 17 is then converted to a compound of Formula (I) as described in Scheme 1 above.

Compounds of Formula (I), where Y and Z are nitrogen, X is CR, where R is cyano, and R¹, R², R³ and R^{3a} are as defined in the Summary of the Invention, can be prepared as described in Schemes 7 and 8 below.

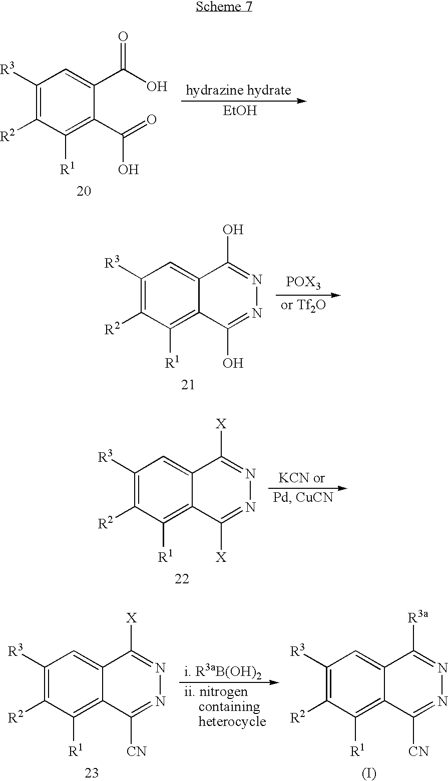

Treatment of compound 20 with hydrazine hydrate in an alcoholic solvent, such as ethanol, and the like, provides 2,4-dihydroxyphthalzine 21. Halogenation of compound 21 with a suitable halogenating agent, such as phosphorus oxychloride or bromide, provides di-halo compound 22, where each X is halo, which, when R² and R³ are the same, is converted to nitrile substituted phthalazine intermediate 23 by reaction with one equivalent of potassium cyanide under nucleophilic conditions, or by palladium catalyzed reaction in the presence of copper cyanide. Alternatively, compound 21 is treated with triflic anhydride to provide a compound 22, where each X is —OTf. The halo or triflate group at C-1 carbon is selectively replace by nitrile by reacting compound 22 with potassium cyanide or copper cyanide in presence of Pd catalyst to provide a compound 23. Compound 23 is then converted to a compound of Formula (I) as described in Scheme 1 above.

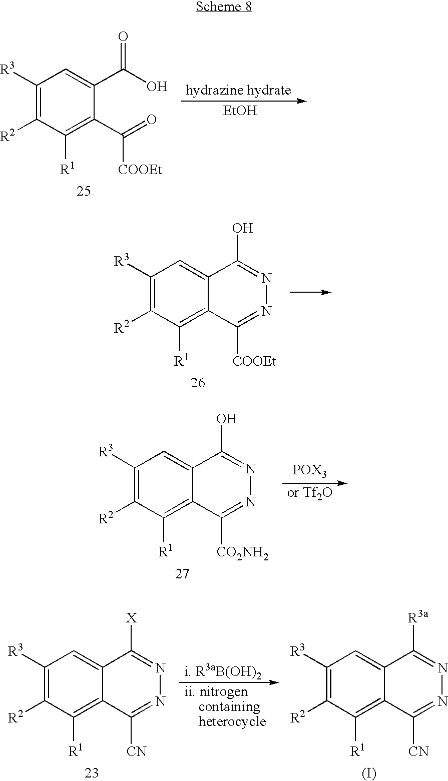

In an alternative method, compound 23 is prepared by cyclization of the oxalate compound 25 (readily produced by Friedel-Crafts acylation) with hydrazine to provide ester 26. Compound 26 is converted to the corresponding amide 27 by standard methods well known in the art. Simple dehydration of 27, concomitant with production of the halo phthalazine under treatment with phosphorous oxyhalide, provides compound 23, which is then converted to a compound of Formula (I) as described above.

### Utility and Methods of Use

The present invention provides methods for treating a disorder or disease by inhibiting PDE10 enzyme. The methods, in general, comprises the step of administering a therapeutically effective amount of a compound of Formula I, or, to a patient in need thereof to treat the disorder or disease.

The compounds of the present invention inhibit PDE10 enzyme activity and hence raise the levels of cAMP or cGMP within cells that express PDE10. Accordingly, inhibition of PDE10 enzyme activity can be useful in the treatment of diseases caused by deficient amounts of cAMP or cGMP in cells. PDE10 inhibitors can also be beneficial in cases wherein raising the amount of cAMP or cGMP above normal levels results in a therapeutic effect. Inhibitors of PDE10 may be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex, and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention are suitable for use in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways (Saxena et al., Br. J. Psychiatry Suppl, 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention are therefore suitable for use in the indication of OCD. OCD may result, in some cases, from streptococcal infections that cause autoimmune reactions in the basal ganglia (Giedd et al., Am J Psychiatry. 157:281-283, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that, since PDE10 degrades cAMP or cGMP, the level of this enzyme affects memory in animals, for example, in humans. A compound that inhibits cAMP phosphodiesterase (PDE) can thereby increase intracellular levels of cAMP, which in turn activate a protein kinase that phosphorylates a transcription factor (cAMP response binding protein). The phosphoylated transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention are suitable for use in treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MC1) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention are suitable for use in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 (also called Machado-Joseph disease or MJD) (ataxin-3); spinocerebellar ataxia type-6 (alpha 1a-voltage dependent calcium channel); spinocerebellar ataxia type-7 (ataxin-7); and spinal and bulbar muscular atrophy (SBMA, also know as Kennedy disease).

The basal ganglia are important for regulating the function of motor neurons; disorders of the basal ganglia result in movement disorders. Most prominent among the movement disorders related to basal ganglia function is Parkinson's disease (Obeso et al., Neurology. 62(1 Suppl 1):S17-30, 2004). Other movement disorders related to dysfunction of the basal ganglia include tardive dyskinesia, progressive supranuclear palsy and cerebral palsy, corticobasal degeneration, multiple system atrophy, Wilson disease, dystonia, tics, and chorea. The compounds of the invention are also suitable for use to treat movement disorders related to dysfunction of basal ganglia neurons.

PDE10 inhibitors are useful in raising cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis.

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity inhibits cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention can be used to stop the growth of cancer cells that express PDE10.

The compounds of the invention are also suitable for use in the treatment of diabetes and related disorders such as obesity, by focusing on regulation of the cAMP signaling system. By inhibiting PDE-10, especially PDE-10A, intracellular levels of cAMP are increased, thereby increasing the release of insulin-containing secretory granules and, therefore, increasing insulin secretion. See, for example, WO 2005/012485, which is hereby incorporated by reference in its entirety. The compounds of Formula (I) can also be used to treat diseases disclosed in US Patent application publication No. 2006/019975, the disclosure of which is incorporated herein by reference in its entirety.

### Testing

The PDE10 inhibitory activities of the compounds of the present invention can be tested, for example, using the in vitro and in vivo assays described in working Biological Examples below.

### Administration and Pharmaceutical Compositions

In general, the compounds of this invention can be administered in a therapeutically effective amount by any of the accepted modes of administration for agents that serve similar utilities. The actual amount of a compound of this invention, i.e., the active ingredient, depends upon numerous factors, such as the severity of the disease to be treated, the age and relative health of the subject, the potency of the compound used, the route and form of administration, and other factors.

Therapeutically effective amounts of compounds of formula (I) may range from approximately 0.05-15 mg per kilogram body weight of the recipient per day (mg/kg/day); or about 0.05-1 mg/kg/day. Thus, for administration to a 70 kg person, the dosage may range from about 0.1 to about 1,000 mg per day, from about 0.5 to 250 mg per day, or from about 3.5 mg to 70 mg per day.

In general, compounds of this invention can be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors, such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, Gennaro, A. R. (Mack Publishing Company, 18th ed., 1995).

The level of the compound in a formulation can vary within the full range employed by those skilled in the art. Typically, the formulation contains, on a weight percent (wt %) basis, from about 0.01-99.99 wt % of a compound of Formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range, and can be administered either simultaneously or sequentially.

Drugs suitable in combination with the compounds of the present invention include, but are not limited to, other suitable schizophrenia drugs such as Clozaril, Zyprexa, Risperidone, and Seroquel; bipolar disorder drugs, including, but not limted to, Lithium, Zyprexa, and Depakote; Parkinson's disease drugs, including, but not limited to, Levodopa, Parlodel, Permax, Mirapex, Tasmar, Contan, Kemadin, Artane, and Cogentin; agents used in the treatment of Alzheimer's disease, including, but not limited to, Reminyl, Cognex, Aricept, Exelon, Akatinol, Neotropin, Eldepryl, Estrogen and Cliquinol; agents used in the treatment of dementia, including, but not limited to, Thioridazine, Haloperidol, Risperidone, Cognex, Aricept, and Exelon; agents used in the treatment of epilepsy, including, but not limited to, Dilantin, Luminol, Tegretol, Depakote, Depakene, Zarontin, Neurontin, Barbita, Solfeton, and Felbatol; agents used in the treatment of multiple sclerosis, including, but not limited to, Detrol, Ditropan XL, OxyContin, Betaseron, Avonex, Azothioprine, Methotrexate, and Copaxone; agents used in the treatment of Huntington's disease, including, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (e.g., sulfonylurea drugs, such as Glyburide, Glimepiride, Chlorpropamide, Tolbutamide, and Glipizide, and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors, and 11beta-HSD inhibitors), hepatic glucose output lowering compounds (such as glucagon antagonists and metaformin, e.g., Glucophage and Glucophage XR), insulin and insulin derivatives (both long and short acting forms and formulations of insulin); and anti-obesity drugs, including, but not limited to, β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat).

### EXAMPLES

The following preparations and examples are given to enable those skilled in the art to more clearly understand and to practice the present invention. They should not be considered as limiting the scope of the invention, but merely as being illustrative and representative thereof.

All NMR spectra were recorded at 300 MHz on a Bruker Instruments NMR unless otherwise stated. Coupling constants (J) are in Hertz (Hz) and peaks are listed relative to TMS (δ 0.00 ppm). Microwave reactions were performed using a Personal Chemistry Optimizer™ microwave reactor in 10 mL Personal Chemistry microwave reactor vials. All microwave reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C18 5 μm column using (i) a gradient of 20/80 (v/v) to 80/20 (v/v) acetonitrile (0.1 v % formic acid)/water (0.1 v % formic acid) over 6 min (Method A), (ii) a gradient of 20/80 (v/v) to 80/20 (v/v) acetonitrile (0.1 v % formic acid)/water (0.1 v % formic acid) over 8 min (Method B), (iii) a gradient of 40/60 (v/v) to 80/20 (v/v) acetonitrile (0.1 v % formic acid)/water (0.1 v % formic acid) over 6 min (Method C), or (iv) a gradient of 40/60 (v/v) to 80/20 (v/v) acetonitrile (0.1 v % formic acid)/water (0.1 v % formic acid) over 8 min (Method D). Preparative HPLC was performed on 30 mm×100 mm Xtera Prep RP₁₈ 5 μm columns using an 8 min gradient of 95/5 (v/v) to 20/80 (v/v) water (0.1 v % formic acid)/acetonitrile (0.1 v % formic acid).

### Synthetic Examples

### Example 1

### Synthesis of 4-bromo-6-ethyl-7-methoxycinnoline

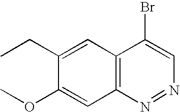

Step 1. Into a 250 mL 3-necked round bottom flask, was placed fuming HNO₃ (20 mL). To this was added concentrated sulfuric acid (28 mL). 1-Ethylbenzene (15 g, 141.51 mmol) was added dropwise with stirring, maintaining the temperature below 95° C. The resulting solution was poured into iced water (200 mL) and the product was extracted using ethyl acetate (3×70 mL). The combined organics were dried over anhydrous MgSO₄ and concentrated. The residue was purified by eluting through a column with a 1:10 (v/v) ethyl acetate/petroleum ether solvent system to afford 22 g (78% yield) of 1-ethyl-2,4-dinitrobenzene as a yellow oil.

Step 2. Iron (25.8 g, 460.71 mmol) was added in several portions to a solution of 1-ethyl-2,4-dinitrobenzene (30 g, 137.76 mmol, prepared as described in Step 1 above) in acetic acid (350 mL), while maintaining the temperature at reflux. The resulting solution was maintained at reflux for a further 10 min. The product was precipitated by the addition of ice, and then extracted with ethyl acetate. The organic layers were combined, dried (anhydrous MgSO₄), filtered, and concentrated. The residue was purified by eluting through a column with a 1:10 (v/v) ethyl acetate/petroleum ether solvent system to afford 12.9 g (51% yield) of 2-ethyl-5-nitro-benzenamine as a brown solid.

Step 3. A solution of sulfuric acid (98%, 39 g, 390.00 mmol) in water (160 mL) was added to 2-ethyl-5-nitrobenzenamine (12.9 g, 69.94 mmol, prepared as described in Step 2 above). The mixture was cooled to 0-5° C., and a solution of sodium nitrite (5.63 g, 81.59 mmol) in water (20 mL) was then added. The resulting solution was maintained for 30 min at 0-5° C. Sulfuric acid (65%, 600 g, 3.98 mol) was then added, and the temperature was maintained at reflux for 1 hr. The reaction mixture was cooled in a bath of iced water, and the product was extracted with ethyl acetate. The organic layers were combined and washed with aqueous saturated sodium bicarbonate and brine (200 mL). The solution was dried (anhydrous MgSO₄), filtered, and concentrated. The residue was purified by eluting through a column with a 1:10 (v/v) ethyl acetate/petroleum ether solvent system to afford 7.65 g (52% yield) of 2-ethyl-5-nitrophenol as a red solid.

Step 4. Potassium carbonate (12.6 g, 91.30 mmol) was added to a solution of 2-ethyl-5-nitrophenol (7.65 g, 36.65 mmol, prepared as described in Step 3 above) in acetone (200 mL). Methyl iodide (19.5 g, 137.32 mmol) was then added, and the resulting solution was maintained at reflux for 3 hr. The solution was allowed to cool, filtered and concentrated. The residue was purified by eluting through a column with a 1:20 (v/v) ethyl acetate/petroleum ether solvent system to afford 5.15 g (70% yiled) of 1-ethyl-2-methoxy-4-nitrobenzene as yellow oil.

Step 5. A mixture of ammonium chloride (15.2 g, 284.11 mmol) in water (100 mL) was added to a solution of 1-ethyl-2-methoxy-4-nitrobenzene (5.15 g, 25.61 mmol, prepared as described above in step 4) in ethanol (100 mL). The mixture was cooled to 0-5° C. and zinc (7.40 g, 113.85 mmol) was added in several portions. Acetic acid (6.83 g, 113.83 mmol) was then added dropwise at 0-5° C. The resulting solution was stirred at room temperature for 3 hr. The mixture was concentrated and sodium bicarbonate was added to adjust the pH to 7. The resulting solution was extracted with ethyl acetate and the organic layers were combined, washed with brine, dried (anhydrous MgSO₄), filtered, and concentrated. The residue was purified by eluting through a column with a 1:5 (v/v) ethyl acetate/petroleum ether solvent system to afford 3.1 g (72% yield) of 4-ethyl-3-methoxy-benzenamine as a green solid.

Step 6. Triethylamine (2.28 g, 22.57 mmol) was added to a solution of 4-ethyl-3-methoxy-benzenamine (3.1 g, 19.50 mmol, prepared as described in Step 5 above) in methylene chloride (100 mL). Acetyl chloride (2.42 g, 30.83 mmol) was then added dropwise at 0-5° C., and the mixture was maintained at this temperature for 30 min. The mixture was concentrated and the product was extracted with ethyl acetate. The organic layers were combined, dried (anhydrous MgSO₄), filtered, and concentrated. The residue was purified by eluting through a column with a 1:2 (v/v) ethyl acetate/petroleum ether solvent system to afford 2.8 g (74%) of N-(4-ethyl-3-methoxyphenyl)acetamide as a pink solid.

Step 7. Aluminum (III) chloride (7.7 g, 58.11 mmol) was added to a solution of N-(4-ethyl-3-methoxyphenyl)acetamide (2.8 g, 13.06 mmol, prepared as described in Step 6 above) in dichloromethane (100 mL). Acetyl chloride (2.3 g, 29.30 mmol) was then added dropwise at 0-5° C., and the resulting solution was maintained at room temperature for 2 hr. Ice (100 g) was added, and the resulting solution was extracted with methylene chloride. The organic layers were combined, washed with saturated sodium bicarbonate and brine, dried (anhydrous MgSO₄), filtered, and concentrated to afford 3.6 g (94% yield) of N-(2-acetyl-4-ethyl-5-methoxyphenyl)acetamide as a red solid.

Step 8. Hydrochloric acid (100 mL) was added to a solution of N-(2-acetyl-4-ethyl-5-methoxyphenyl)acetamide (3.6 g, 12.26 mmol, prepared as described in Step 7 above) in 1,4-dioxane (100 mL). The resulting solution was maintained at 85° C. for 3 hr. The mixture was concentrated and sodium bicarbonate was added to adjust the pH of the solution to 7. The product was extracted with ethyl acetate. The organic layers were combined, washed with brine, dried (anhydrous MgSO₄), filtered, and concentrated. The residue was purified by eluting through a column with a 1:20 (v/v) ethyl acetate/petroleum ether solvent system to afford 1.8 g (68% yield) of 1-(2-amino-5-ethyl-4-methoxy phenyl)ethanone as a light yellow solid.

Step 9. A solution of sodium nitrite (380 mg, 5.51 mmol) in water (5 mL) was added dropwise to a chilled solution of 1-(2-amino-5-ethyl-4-methoxyphenyl)ethanone (1 g, 4.66 mmol, prepared as described above in Step 8) in 12 M hydrochloric acid (50 mL) at 0-5° C. The resulting solution was maintained at room temperature for 16 hr. The pH of the mixture was adjusted to 7 by the addition of sodium bicarbonate. The product was extracted with ethyl acetate and the combined organics were washed with brine, dried (anhydrous MgSO₄), filtered, and concentrated to afford 400 mg (38% yield) of 6-ethyl-7-methoxycinnolin-4-ol as a pink solid.

Step 10. Phosphoryl tribromide (2.1 g, 7.32 mmol) was added to a solution of 6-ethyl-7-methoxycinnolin-4-ol (480 mg, 2.12 mmol, prepared as described in Step 9 above) in acetonitrile (100 mL) and the resulting solution was maintained at 70° C. for 3 hr. The pH of the mixture was adjusted to 7 by the addition of sodium bicarbonate. The mixture was concentrated and the product was extracted with ethyl acetate (3×100 mL). The organic layers were combined, washed with brine (1×50 mL), dried (anhydrous MgSO₄), filtered, and concentrated. The residue was purified by eluting through a column with a 1:2 (v/v) ethyl acetate/petroleum ether solvent system to afford 200 mg (30% yield) of 4-bromo-6-ethyl-7-methoxycinnoline as a pink solid. ¹H NMR δ 9.31 (1H, s), 7.82 (1H, s), 7.77 (1H, s), 4.08 (3H, s), 2.85-2.93 (2H, q), and 1.26-1.37 (3H, t).

### Example 2

### Synthesis of 4-bromo-7-ethyl-6-methoxycinnoline

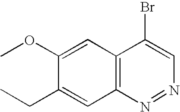

Step 1. Aluminum (III) chloride (27 g, 202.49 mmol) was added to a chilled solution of 1-ethylbenzene (10.6 g, 99.85 mmol) in methylene chloride (100 mL) at −70° C. A solution of acetic anhydride (10.2 g, 99.91 mmol) in methylene chloride (20 mL) was added dropwise over 3 hr, while maintaining the temperature at −70° C. The resulting solution was maintained for 2 hr between −70 and −50° C., then added to a mixture of ice and hydrochloric acid (100 mL). The product was extracted with methylene chloride and the organic, dried, filtered, and concentrated to afford 15 g (86% yield) of 1-(4-ethylphenyl)ethanone as a colorless liquid.

Step 2. 1-(4-Ethylphenyl)ethanone (15 g, 86.03 mmol, prepared as described in Step 1 above) was added dropwise to chilled concentrated sulfuric acid (20 mL) at 0-5° C. A solution of fuming nitric acid (8.1 g) in concentrated sulfuric acid (10 mL) was added dropwise and the mixture was maintained for 15 min at 0-5° C., then added slowly to 300 mL iced water. The product was extracted with methylene chloride. The organic layers were combined, washed with saturated sodium bicarbonate and brine, dried, filtered, and concentrated. The residue was purified by eluting through a column with a 1:50 (v/v) ethyl acetate/petroleum ether solvent system to afford 14 g (84% yield) of 1-(4-ethyl-3-nitrophenyl)ethanone as a yellow liquid.

Step 3. A solution of 1-(4-ethyl-3-nitrophenyl)ethanone (10 g, 49.17 mmol, prepared as described in Step 2 above) in acetic acid (10 mL) was added in several portions to a mixture of iron (8.2 g, 146.82 mmol) in water (100 mL), while warming the mixture to a temperature of 80-90° C. The resulting solution was maintained at reflux for 1.5 hr. The mixture was adjusted to pH 7-8 by the addition of ammonia (28%) and was filtered. The product was extracted with methylene chloride and the organic layers were combined, washed with brine, dried (anhydrous Na₂SO₄), filtered, and concentrated to afford 8.6 g (91% yield) of 1-(3-amino-4-ethylphenyl)ethanone as a yellow liquid.

Step 4. 1-(3-Amino-4-ethylphenyl)ethanone (8.6 g, 44.79 mmol, prepared as described in Step 3 above) was added to chilled sulfuric acid (20%, 80 mL) at 0° C. Sodium nitrite (4.5 g, 65.22 mmol) in water (20 mL) was then added dropwise maintaining a temperature of 0-5° C. The resulting solution was allowed to react for 1 hr at 0-5° C. Urea (1.6 g, 26.64 mmol) was then added and the resulting solution was maintained for 15 min at 0-5° C. This solution was then added dropwise to 30% sulfuric acid (100 mL), while heating to a temperature of 100° C. The resulting solution was maintained at 100° C. for a further 15 min, and then cooled and filtered. The filter cake was washed with 10% sodium bicarbonate. The solid was dried to afford 6.8 g (88% yield) of 1-(4-ethyl-3-hydroxyphenyl)ethanone as a yellow solid.

Step 5. Propan-2-one (50 mL) and potassium carbonate (8.3 g, 60.14 mmol) were added to 1-(4-ethyl-3-hydroxyphenyl)ethanone (6.6 g, 38.23 mmol, prepared as described in Step 4 above). Methyl iodide (17.1 g, 120.42 mmol) was then added and the resulting solution was maintained at 60° C. for 3 hr. The mixture was concentrated and diluted with water (100 mL). The product was extracted with methylene chloride. The organic layers were combined and dried over anhydrous Na₂SO₄. The residue was purified by eluting through a column with a 1:20 (v/v) ethyl acetate/petroleum ether solvent system to afford 7 g (94% yield) of 1-(4-ethyl-3-methoxyphenyl)ethanone as a yellow liquid.

Step 6. Acetic acid (1 mL) was added to 1-(4-ethyl-3-methoxyphenyl)ethanone (300 mg, 1.69 mmol, prepared as described in Step 5 above). The mixture was chilled to 0-5° C. and fuming nitric acid (1 mL) was added. The resulting was maintained at room temperature for 2 hr, and then cooled in iced water. The product was extracted with methylene chloride. The organic layers were combined, washed with 10% sodium bicarbonate solution, and brine, dried (anhydrous Na₂SO₄), filtered, and concentrated. The residue was purified by eluting through a column with a 1:50 (v/v) ethyl acetate/petroleum ether solvent system to afford 100 mg (27% yield) of 1-(4-ethyl-5-methoxy-2-nitrophenyl)-ethanone as a yellow solid.

Step 7. A solution of 1-(4-ethyl-5-methoxy-2-nitrophenyl)ethanone (250 mg, 1.12 mmol, prepared as described above in Step 6) in acetic acid (2 mL) was added to a mixture of iron (200 mg, 3.58 mmol) in water (30 mL). The resulting mixture was heated to reflux temperature for 45 min. The pH was adjusted to 8 by the addition of ammonia (28%) and the mixture was filtered. The product was extracted with ethyl acetate and the organic layers were combined, dried (anhydrous Na₂SO₄), and concentrated to afford 200 mg (88% yield) of 1-(2-amino-4-ethyl-5-methoxyphenyl)ethanone as a yellow liquid.

Step 8. Sodium nitrite (250 mg, 3.62 mmol) in water (5 mL) was added to a chilled solution of 1-(2-amino-4-ethyl-5-methoxyphenyl)ethanone (500 mg, 2.46 mmol, prepared as described above in Step 7) in concentrated hydrochloric acid (10 mL) at 0-5° C. The resulting solution was maintained at 0-5° C. for 15 min. Iced water (50 mL) was then added, and the pH was adjusted to 6-7 by the addition of sodium carbonate solution (10%). The product was extracted with ethyl acetate and the organic layers were combined, dried (anhydrous Na₂SO₄), and concentrated. The residue was purified by eluting through a column with a 1:1 (v/v) ethyl acetate/petroleum ether solvent system to afford 300 mg (60%) of 7-ethyl-6-methoxycinnolin-4-ol as a brown solid.

Step 9. Phosphoryl tribromide (1.4 g, 4.88 mmol) was added to a solution of 7-ethyl-6-methoxycinnolin-4-ol (300 mg, 1.47 mmol, prepared as described above in Step 8) in acetonitrile (20 mL) and the resulting solution was maintained at 70° C. for 3 hr. Iced water (30 mL) was then added. The pH was adjusted to 6-7 by the addition of sodium carbonate (10% solution) and the product was extracted with ethyl acetate. The organic layers were combined, dried (anhydrous Na₂SO₄), filtered, and concentrated. The residue was purified by eluting through a column with a 1:8 (v/v) ethyl acetate/petroleum ether solvent system to afford 150 mg (38% yield) of 4-bromo-7-ethyl-6-methoxycinnoline as a light yellow solid. ¹H NMR (400 MHz, CDCl₃) δ 1.36 (t, 3H), 2.88 (q, 2H), 4.08 (s, 3H), 7.21 (s, 1H), 8.29 (s, 1H), and 9.31 (s, 1H).

### Example 3

### Synthesis of 4-bromo-6,7-dimethoxyphthalazine

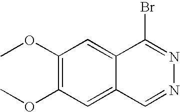

Step 1. Into a 500 mL round bottom flask containing a solution of 3,4-dimethoxybenzoic acid (18.2 g, 99.91 mmol) in AcOH (250 mL) was added Br₂ (17.6 g, 110.00 mmol). The resulting solution was stirred for 3 days while the temperature was maintained at 50° C. The reaction mixture was cooled to room temperature and the solid product was collected by filtration. The filter cake was washed with hexanes and dried to provide 10 g of crude 2-bromo-4,5-dimethoxybenzoic acid as a white solid.

Step 2. Into a 250 mL 3-necked round bottom flask purged and maintained with an inert atmosphere of nitrogen, was added a solution of n-BuLi (2.5M, 36 mL) in THF (120 mL) and cooled to −78° C. A solution of 2-bromo-4,5-dimethoxybenzoic acid (7.8 g, 129.88 mmol) in THF (60 mL) was added dropwise with stirring over 30 min. The mixture was stirred for another 30 min at −78° C., followed by the addition of DMF (2.7 g, 36.94 mmol). The resulting solution was stirred for 1 hr, while the temperature was allowed to warm to room temperature. Reaction progress was monitored by TLC (CH₂Cl₂/MeOH (15:1, v/v)) and upon completion the mixture was concentrated, diluted with H₂O and extracted with CH₂Cl₂. The combined organic fractions were washed with 150 mL of brine, dried over anhydrous Na₂SO₄, and concentrated to provide 5 g (80%) of 2-formyl-4,5-dimethoxybenzoic acid as a orange solid.

Step 3. Into a 500 mL round bottom flask was added 2-formyl-4,5-dimethoxybenzoic acid (5 g, 11.89 mmol), N₂H₄—H₂O (20 mL), and MeOH (300 mL). The resulting solution was warmed to reflux with stirring for 5 hr. Reaction progress was monitored by TLC(CH₂Cl₂/MeOH (15:1, v/v)) and upon completion the mixture was cooled to room temperature and concentrated. The residue was dissolved in 200 mL of H₂O and the pH was adjusted to 10 by the addition of NaOH (2N). The resulting solution was extracted with CH₂Cl₂ and the combined organic fractions were dried over anhydrous Na₂SO₄ and concentrated to provide 900 mg (37%) of 6,7-dimethoxyphthalazin-1(2H)-one as a white solid.

Step 4. Into a 250 mL round bottom flask was added 6,7-dimethoxyphthalazin-1(2H)-one (900 mg, 4.36 mmol), CH₃CN (150 mL), and POBr₃ (6.27 g, 21.85 mmol). The resulting mixture was warmed to reflux with stirring for 4 hr and the reaction progress was monitored by TLC (CH₂Cl₂/MeOH (15:1, v/v)). The mixture was concentrated (rotary evaporator) and the residue was dissolved in 200 mL of H₂O. The pH was adjusted to 10 by the addition of NaOH (2N) and then extracted with 3×100 mL of CH₂Cl₂. The combined organic fractions were dried over anhydrous Na₂SO₄, concentrated, and purified by silica gel chromatography using 50:1 (v/v) CH₂Cl₂/MeOH as eluant to provide 650 mg (55%) of 1-bromo-6,7-dimethoxyphthalazine as a white solid.

### Example 4

### Synthesis of 4-bromo-7-fluoro-6-methoxycinnoline

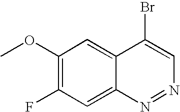

Step 1. 1-(4-Fluoro-3-nitrophenyl)ethanone: Into a 500 mL 3-necked round bottom flask containing conc. sulfuric acid (166 mL) was added 1-(4-fluorophenyl)ethanone (74.6 g, 540.58 mmol) dropwise over a time period of 30 min with stirring, while maintaining the temperature between −10 and 0° C. This was followed by the dropwise addition of a solution of nitric acid (65%) (43 g, 648.41 mmol) in H₂SO₄ (98%) (60 mL) over a time period of 30 min, while maintaining the temperature from −10 to 0° C. The resulting solution was stirred for 7 hr at −10 to 0° C. and the reaction progress was monitored by TLC (EtOAc/PE (1:1, v/v)). Upon completion a mixture of ice and water was added to quench and the resulting solution was extracted with 3×100 mL of DCM. The organic layers were combined and washed with 3×200 mL of NaHCO₃, brine, H₂O, dried (anhydrous Na₂SO₄), and concentrated. The material was purified by silica gel chromatography using a gradient elution going from 100:1 (v/v) to 5:1 (v/v) EtOAc/PE to provide 43 g (44%) of 1-(4-fluoro-3-nitrophenyl)ethanone as a yellow solid.

Step 2. Into a 2 L round bottom flask containing a solution of 1-(4-fluoro-3-nitrophenyl)ethanone (40 g, 218.58 mmol) in CH₃OH (900 mL) was added ammonium chloride (550 mL), and iron (31.8 g, 567.86 mmol) in several portions. The resulting mixture was warmed to reflux for 3 hr. Reaction progress was monitored by TLC (PE:EtOAc (1:1, v/v)) and upon completion, the mixture was cooled to room temperature, filtered, and concentrated. The residue was dissolved in DCM, and the resulting mixture was washed with brine, H₂O, dried (anhydrous Na₂SO₄), and concentrated to provide 26.2 g of 1-(3-amino-4-fluorophenyl)ethanone as a brown liquid.

Step 3. Into a 2L 3-necked round bottom flask was added 1-(3-amino-4-fluorophenyl)ethanone (13.6 g, 88.89 mmol), 230 mL of 35% sulfuric acid in water, and 160 mL of H₂O. This was followed by the dropwise addition of a solution of sodium nitrate (6.2 g, 89.86 mmol) in water (30 mL) with stirring, while maintaining the temperature between −5 and 0° C. This was followed by the addition of a solution of cupric nitrate (300 g, 1.24 mol) in water (800 mL) and then cuprous oxide (30 g, 209.79 mmol). The resulting solution was stirred at room temperature for 15 min and the reaction progress was monitored by TLC (EtOAc/PE (1:1, v/v)). The reaction mixture was filtered and the filtrate was extracted with DCM. The combined organic layers were dried (anhydrous Na₂SO₄) and concentrated (rotary evaporator). The residue was purified by silica gel chromatography using a gradient elution of petroleum ether/EtOAc going from 30:1 (v/v) to 10:1 (v/v) to provide 2.6 g (19%) of 1-(4-fluoro-3-hydroxyphenyl)ethanone as a pale yellow solid.

Step 4. Into a 50 mL round bottom flask was added 1-(4-fluoro-3-hydroxyphenyl)ethanone (2.7 g, 17.53 mmol), DMF (30 mL), sodium carbonate (5 g, 47.17 mmol), and iodomethane (5 g, 35.21 mmol). The solution was stirred overnight at 90° C. The reaction progress was monitored by TLC (EtOAc/P (1:1, v/v)). The mixture was concentrated by evaporation under vacuum using a rotary evaporator and the residue was dissolved in 100 mL of DCM and 100 mL of H₂O. The organic phase was separated and washed with H₂O, dried over anhydrous Na₂SO₄, and concentrated by evaporation under vacuum using a rotary evaporator to provide 2.9 g (92%) of 1-(4-fluoro-3-methoxyphenyl)ethanone as a light yellow solid.

Step 5. 1-(4-Fluoro-3-methoxyphenyl)ethanone (2.4 g, 14.29 mmol) was added with stirring to a 250 mL 3-necked round bottom flask containing a solution of fuming nitric acid (56 g, 844.44 mmol) in H₂O (9.3 g) at −40 to −30° C. The solution was stirred for 6 hr while maintaining the temperature below −30° C. and the reaction progress was monitored by TLC (Et₂O/petroleum ether (2:1, v/v)). Upon reaction completion the temperature of the mixture was cooled to below −40° C. and 100 mL of cold water was added. The mixture was filtered and the filter cake was washed with H₂O. The solid was dried in an oven under reduced pressure to provide 2 g (65.7%) of (1-(4-fluoro-5-methoxy-2-nitrophenyl)ethanone as a yellow solid.

Step 6. Into a 50 mL round bottom flask was added a solution of 1-(4-fluoro-5-methoxy-2-nitrophenyl)ethanone (1 g, 4.69 mmol), methanol (30 mL), and tin(II) chloride dihydrate (5 g, 22.16 mmol). The reaction mixture was warmed to reflux with stirring overnight and the reaction progress was monitored by TLC (Et₂O/PE (1:2, v/v)). The mixture was concentrated and the residue was dissolved in 50 mL of Et₂O, washed with 3×50 mL of saturated aqueous NaHCO₃, and concentrated. The residue was purified by silica gel chromatography using 20:1 (v/v) PE:Et₂O as eluant to provide 400 mg (47%) of 1-(2-amino-4-fluoro-5-methoxyphenyl)ethanone as a yellow solid.

Step 7. Into a 100 mL 3-necked round bottom flask was added 1-(2-amino-4-fluoro-5-methoxyphenyl)ethanone (300 mg, 1.64 mmol) and 1 M HCl (50 mL). Insoluble impurities were removed by filtration and then a solution of sodium nitrite (200 mg, 2.90 mmol) in 1 mL of H₂O was added. The reaction was stirred for two hr at −5° C., followed by the addition of urea (500 mg, 8.33 mmol). Stirring continued for 30 min and then the temperature was raised and maintained at 80° C. overnight. The reaction mixture was cooled in an ice-water bath and the product collected by filtration to provide 170 mg (53%) of 7-fluoro-6-methoxycinnolin-4-ol as a brick red solid.

Step 8. Into a 50 mL round bottom flask was added 7-fluoro-6-methoxycinnolin-4-ol (170 mg, 0.88 mmol), CHCl₃ (15 mL), and phosphorus oxybromide (1.7 g, 5.99 mmol). The reaction mixture was stirred overnight at reflux and the reaction progress was monitored by TLC (CH₂Cl₂/MeOH (10/1, v/v)). The reaction mixture was quenched with water and the pH was adjusted to 7 by the addition of NaHCO₃ powder. The mixture was concentrated (rotary evaporator) and the product was isolated by filtration. The filter cake was washed with H₂O to provide, after drying, 200 mg (89%) of 4-bromo-7-fluoro-6-methoxycinnoline as a brown solid. ¹H NMR: (DMSO) δ 9.57(s 1H), 7.42 (d 1H), 8.36 (d 1H), and 4.13(d 2H).

### Example 5

### Synthesis of N-cyclopropyl-5-(7-ethyl-6-methoxycinnolin-4-yl)-4,5,6,7-tetrahydro-1H-pyrazolo[4,3-c]pyridine-3-carboxamide hydroformate

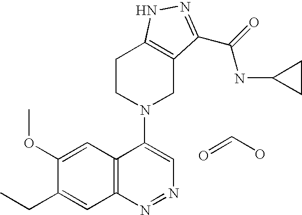

4-Bromo-7-ethyl-6-methoxycinnoline (20 mg, 0.08 mmol, prepared as described in Example 2 above), N-cyclopropyl-4,5,6,7-tetrahydro-1H-pyrazolo[4,3-c]pyridine-3-carboxamide trifluoroacetate (20 mg, 0.06 mmol), toluene (0.3 mL), tris(dibenzylideneacetone)dipalladium(0) (1.8 mg, 0.0020 mmol), 9,9-dimethyl-4,5-bis(diphenylphosphino)xanthane (2.83 mg, 0.005 mmol), and sodium tert-butoxide (14 mg, 0.15 mmol) were added to a 10 mL microwave tube and the mixture was subjected to microwave irradiation at 140° C. for 3 min. The mixture was filtered through celite, which was washed with methanol and methylene chloride, and the combined organic filtrates were concentrated. The residue was purified by preparative HPLC (using a gradient of 20-80 v % acetonitrile in water (with 0.1 v % formic acid)) to afford 4 mg (20% yield) of N-cyclopropyl-5-(7-ethyl-6-methoxycinnolin-4-yl)-4,5,6,7-tetrahydro-1H-pyrazolo[4,3-c]pyridine-3-carboxamide hydroformate as a yellow solid. LC/MS (EI) t_{R} 2.55 min (Method A), m/z 393 (M⁺+1).

### Example 6

### Synthesis of 4-(6,7-dimethoxy-3,4-dihydroisoquinolin-2(1H)-yl)-7-ethyl-6-methoxycinnoline hydroformate

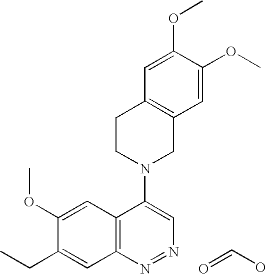

Proceeding as described in Example 5 and using 6,7-dimethoxy-1,2,3,4-tetrahydroisoquinoline, 4-(6,7-dimethoxy-3,4-dihydroisoquinolin-2(1H)-yl)-7-ethyl-6-methoxycinnoline hydroformate was prepared (2.5 mg, 4.8% yield). LC/MS (EI) t_{R} 4.04 min (Method A), m/z 380 (M⁺+1).

### Example 7

### Synthesis of 2-(7-ethyl-6-methoxycinnolin-4-yl)-7-methoxy-3,4-dihydroisoquinolin-1(2H)-one hydroformate

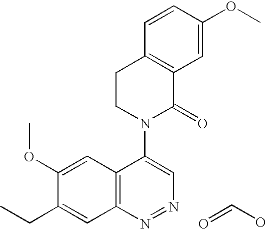

4-Bromo-7-ethyl-6-methoxycinnoline (40 mg, 0.0001 mol, prepared as described in Example 2 above), toluene (1 mL), 7-methoxy-3,4-dihydro-2H-isoquinolin-1-one (31.8 mg, 0.180 mmol), copper(I) iodide (5 mg, 0.03 mmol), potassium carbonate (41.4 mg, 0.299 mmol), and N,N′-dimethyl-1,2-ethanediamine (10 μL) were added to a 10 mL microwave tube and the mixture was subjected to microwave irradiation at 140° C. for 3 min. Additional N,N′-Dimethyl-1,2-ethanediamine (50 μL) was added, and the mixture was heated to 115° C. for 18 h. After cooling to room temperature, the mixture was filtered through celite, which was washed with methylene chloride, and the combined organic filtrates were concentrated. The product was purified by preparative HPLC using a gradient of 20-80 v % acetonitrile (0.1 v % formic acid) to afford 25 mg (40% yield) of 2-(7-ethyl-6-methoxycinnolin-4-yl)-7-methoxy-3,4-dihydroisoquinolin-1(2H)-one hydroformate as a yellow solid. LC/MS (EI) t_{R} 6.95 min (Method A), m/z 364 (M⁺+1).

### Example 8

### Synthesis of 2-(6-ethyl-7-methoxycinnolin-4-yl)-7-methoxy-3,4-dihydroisoquinolin-1(2H)-one hydroformate

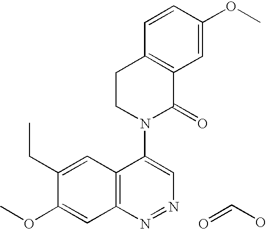

Proceeding as described in Example 7 and using 7-methoxy-3,4-dihydro-2H-isoquinoline, 2-(6-ethyl-7-methoxycinnolin-4-yl)-7-methoxy-3,4-dihydroisoquinolin-1(2H)-one hydroformate was prepared 2.5 mg (4% yield). LC/MS (EI) t_{R} 7.05 min (Method A), m/z 364 (M⁺+1).

### Example 9

### Synthesis of 4-(1-benzyl-1H-pyrazol-4-yl)-7-ethyl-6-methoxycinnoline hydroformate

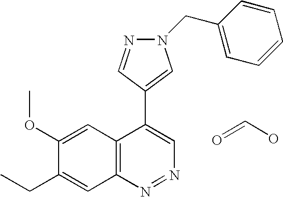

A mixture of 4-bromo-7-ethyl-6-methoxycinnoline (50 mg, 0.0002 mol, prepared as described in Example 2 above), bis(triphenylphosphine)palladium(II) chloride (23.0 mg, 0.0328 mmol), 1-benzyl-1H-pyrazole-4-boronic acid (40 mg, 0.0002 mol), sodium carbonate in water (2.00 M, 0.067 mL), and 1,2-dimethoxyethane:water:ethanol (7:3:2 (v/v/v), 1 mL) was added to a microwave tube and the mixture was subjected to microwave irradiation at 140° C. for 3 min. The mixture was filtered through celite, which was washed with methanol and methylene chloride and the combined organic filtrates were concentrated. The residue was purified by preparative HPLC (using a gradient of 20-80 v % acetonitrile:water (with 0.1 v % formic acid)) to afford 26 mg (40% yield) of 4-(1-benzyl-1H-pyrazol-4-yl)-7-ethyl-6-methoxycinnoline hydroformate as a tan solid. LC/MS (EI) t_{R} 7.2 min (Method A), m/z 345 (M⁺+1).

### Example 10

### Synthesis of 1′-(6,7-dimethoxyphthalazin-1-yl)-1,3′-bipiperidin-2-one

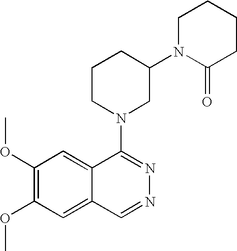

Into a flame-dried 5 mL microwave tube under argon was added 1-bromo-6,7-dimethoxyphthalazine (49.9 mg, 0.185 mmol), 3-(N-delta-valerolactam)piperidine hydrochloride (50.9 mg, 0.233 mmol), tris(dibenzylideneacetone)dipalladium(0) (9.0 mg, 0.0098 mmol), 9,9-dimethyl-4,5-bis(diphenylphosphino)xanthene (11.5 mg, 0.0199 mol), sodium tert-butoxide (44.1 mg, 0.459 mmol), and toluene (0.5 mL, 4.0 mmol). The resulting yellow suspension was stirred at 50° C. overnight and reaction progress was monitored by LC/MS. Tetrahydrofuran (0.1 mL) was added to the reaction mixture to help dissolve the starting phthalazine and then the mixture was irradiated in the microwave at 140° C. for 5 min. The mixture was loaded onto a 1.54 g SCX column and rinsed with methanol (30 mL, 0.7 mol) and the product was eluted with 2.0 M ammonia in methanol (15 mL). The mixture was concentrated and the product was purified on a C18 preparative HPLC column (30×100 mm) using CH₃CN:H₂O (with 0.1 v % formic acid) with a gradient from 20 v % CH₃CN to 80 v % CH₃CN over 8 minutes at a flow rate of 45 mL/min. Detection was performed at a wavelength of 335 nm and the product had a retention time of 1.7 min. This material was loaded onto an SCX column (0.83 g) and washed with one column volume of MeOH and then eluted with 2.0 M ammonia in MeOH (10 mL). Solvent was removed under reduced pressure (rotovap) to provide 4.2 mg of 1′-(6,7-dimethoxyphthalazin-1-yl)-1,3′-bipiperidin-2-one as a white solid. LC/MS: m/z 371.2 (M⁺+1).

### Example 11

### Synthesis of methyl 2-(6,7,8-trimethoxyquinazoline-4-yl)-1,2,3,4-tetrahycroisoquinoline-8-carboxylate

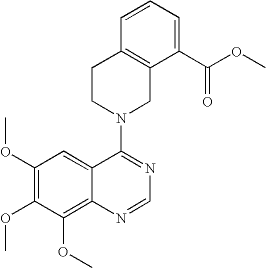

Methyl 1,2,3,4-tetrahydroisoquinoline-8-carboxylate hydrochloride (268 mg, 1.18 mmol), N,N-dimethylacetamide (14.6 mL, 0.158 mol), 4-chloro-6,7,8-trimethoxyquinazoline (300 mg, 1.18 mol), sodium iodide (80 mg, 0.0005 mol), and potassium carbonate (407 mg, 02.94 mol) were combined and heated at 160° C. for 12 hr. The crude product was purified by preparative HPLC with a C18 column using acetonitrile:water (with 0.1% formic acid) as eluant with a gradient from 10:90 (v/v) to 80:20 (v/v) at a flow rate of 45 mL/min to give methyl 2-(6,7,8-trimethoxyquinazoline-4-yl)-1,2,3,4-tetrahycroisoquinoline-8-carboxylate.

### Example 12

### Synthesis of 7-fluoro-6-methoxy-4-[2-(4-methoxyphenyl)morpholin-4-yl]cinnoline

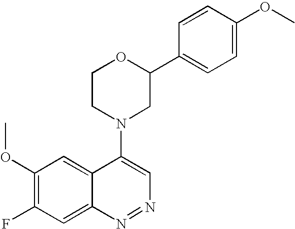

Into a 25 mL round-bottom flask was added 4-bromo-7-fluoro-6-methoxycinnoline (50 mg, 0.2 mmol), 2-(4-methoxyphenyl)morpholine (28 mg, 00.15 mmol), tris(dibenzylideneacetone)-dipalladium(0) (7.05 mg, 7.70 μmol), 9,9-dimethyl-4,5-bis(diphenylphosphino)xanthene (8.59 mg, 0.0148 mmol), sodium tert-butoxide (21.33 mg, 0.2220 mmol), and toluene (0.4 mL, 4 mmol). The resulting yellow-brown suspension was warmed to 55° C. with stirring for 24 hr, adhered to an SCX column with MeOH, and the crude product eluted with 7.0 M ammonia in methanol. Purification by rotary chromatography using a gradient elution from 100% chloroform to 10 v % MeOH in chloroform provided 16 mg of 7-fluoro-6-methoxy-4-[2-(4-methoxyphenyl)morpholin-4-yl]cinnoline as a reddish foam.

### Example 13

### Synthesis of 4-(3-(3,5-dimethoxyphenyl)piperazin-1-yl)-6,7,8-trimethoxycinnoline formate

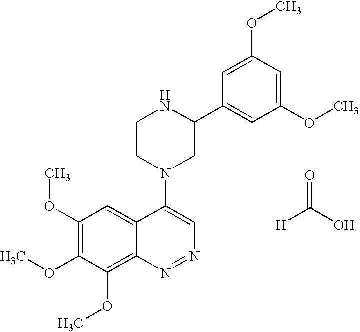

The title compound was synthesized following procedures slightly modified from those described above.

### Biological Examples

### Example 14

### mPDE10A7 Enzyme Activity and Inhibition

Enzyme Activity. To analyze the enzyme activity, 5 μL of serial diluted mPDE10A7 containing lysate were incubated with equal volumes of diluted (100-fold) fluorescein labeled cAMP or cGMP for 30 min in MDC HE 96-well assay plates (Molecular Devices Corp., Sunnyvale Calif.) at room temperature. Both the enzyme and the substrates were diluted in the following assay buffer: Tris/HCl (pH 8.0) 50 mM, MgCl₂ 5 mM, 2-mercaptoethanol 4 mM, and BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP assay kit (Molecular Devices) was used to assess enzyme properties of mPDE10A7. Data were analyzed with SOFTMAX PRO software (Molecular Devices).

Enzyme Inhibition. To check the inhibition profile, 10 μL of serial diluted compounds were incubated with 30 μl of diluted PDE enzymes in a 96-well polystyrene assay plate for 30 min at room temperature. After incubation, 5 μL of the compound-enzyme mixture were aliquoted into a MDC HE black plate, mixed with 5 μL of 100-fold diluted fluorescein labeled substrates (cAMP or CGMP), and incubated for 30 min at room temperature. The reaction was stopped by adding 20 μL of diluted binding reagents and counted in an Analyst GT for fluorescence polarization. The data were analyzed with SoftMax Pro. Exemplar compounds of the invention show activities with IC₅₀ values of generally less than 5 μM.

### Example 15

### Apomorphine Induced Deficits in Prepulse Inhibition of the Startle Response in Rats, an In Vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g., the reflex activity of the rats is measured. The reflex of the rats to the burst of noise may be attenuated by preceding the startle stimulus with a stimulus of lower intensity, at 3 to 12 db above background (65 db), which attenuates the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine reduces the inhibition of the startle reflex produced by the prepulse. Antipsychotic drugs such as haloperidol prevents apomorphine from reducing the prepulse inhibition of the startle reflex. This assay may be used to test the antipsychotic efficacy of PDE10 inhibitors, as they reduce the apomorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 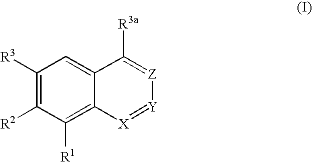
or an individual stereoisomer, mixtures of stereoisomers, or a pharmaceutically acceptable salt thereof, wherein:
Y and Z are nitrogen and X is —CR═, wherein R is hydrogen, alkyl, cyano, or halo; X and Y are nitrogen and Z is ═CH—; or X and Z are nitrogen and Y is ═CH—;
R¹, R², and R³ are independently hydrogen, alkyl, alkoxy, halo, haloalkyl, haloalkoxy, cyano, hydroxy, carboxy, alkoxycarbonyl, amino, alkylamino, dialkylamino, alkylcarbonyl, or cycloalkyl; provided that at least one of R¹, R², and R³ is not hydrogen, and provided that when X and Y or X and Z are nitrogen, and R¹ is hydrogen, then R² and R³ are not both independently hydroxy, alkoxy, or haloalkoxy; and
R^{3a} is aryl, heteroaryl, or heterocyclyl ring substituted with:
R⁴, where R⁴ is hydrogen; alkyl; halo; haloalkyl; haloalkoxy; cycloalkyl; cycloalkylalkyl; aryl; heteroaryl; heterocyclyl; aralkyl; heteroaralkyl; heterocyclylalkyl; or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶, where R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; provided that at least one of R⁴, R⁵ and R⁶ is not hydrogen;
wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e}, where R^{d} and R^{e} are chloro or fluoro;
provided that:
(i) when R^{3a} is pyrrolidin-1-yl, then R⁴ is not —OR⁷, where R⁷ is substituted or unsubstituted aryl or heteroaryl;
(ii) when X and Y or X and Z are nitrogen, R^{3a} is piperidin-1-yl, one of R⁵ and R⁶ is hydrogen, and R⁴ is substituted or unsubstituted aryl or heteroaryl, then the other of R⁵ and R⁶ is not hydrogen, alkyl, carboxy, alkoxycarbonyl, cyano, hydroxyl, alkoxy, —COR, —CONRR′, or —NRR′, where R and R′ are independently hydrogen, alkyl, or unsubstituted aryl, or —NHCOR, where R is alkyl or unsubstituted aryl;
(iii) when X and Y or X and Z are nitrogen, R^{3a} is piperidin-1-yl, both of R⁵ and R⁶ are hydrogen, or one of R⁵ and R⁶ is hydrogen and the other of R⁵ and R⁶ is substituted or unsubstituted aryl or heteroaryl, then R⁴ is not hydrogen, alkyl, —COR⁷ (where R⁷ is unsubstituted aryl), —COOR⁷ (where R⁷ is unsubstituted aryl), —CONR⁷R⁹, —NR⁷R¹⁰, or —NHCOR⁷ (where R⁹ and R¹⁰ are is hydrogen, alkyl, or unsubstituted aryl; and each R⁷ is unsubstituted aryl);
(iv) when X and Y are nitrogen, two of R¹, R², and R³ are hydrogen and the other of R¹, R², and R³ is alkyl or halo, and R^{3a} is aryl, then (a) when two of R⁴, R⁵ and R⁶ are hydrogen, then the other of R⁴, R⁵ and R⁶ is not alkyl, halo, hydroxy, —COR′ (where R′ is alkyl), or —OC(O)R′ or —SO₂R′ (where R′ is aryl optionally substituted with alkyl); and (b) when one of R⁴, R⁵ and R⁶ is hydrogen, then the other two of R⁴, R⁵ and R⁶ are not independently selected from alkyl, hydroxy, or —OCOR′, where R′ is aryl;
(v) when X and Y are nitrogen, two of R¹, R² and R³ are hydrogen and the other of R¹, R², and R³ is chloro, then R^{3a} is not indolin-1-yl or indol-1-yl, each substituted with alkyl and alkoxy and a third substituent selected from —CH₂—C(O)—OR, wherein R′ is hydrogen or methyl;
(vi) when X and Z or Y and Z are nitrogen, then R^{3a} is not:
(a) substituted or unsubstituted 1,2,3,4-tetrahydroquinolinyl;
(b) indolin-1-yl substituted with R⁴, R⁵ and R⁶, where two of R⁴, R⁵ and R⁶ are hydrogen and the other of R⁴, R⁵ and R⁶ is halo;
(c) piperidin-1-yl substituted with R⁴, R⁵ and R⁶, where two of R⁴, R⁵ and R⁶ are hydrogen and the other of R⁴, R⁵ and R⁶ is
quinazoline-2,4(1H,3H)-dione or quinazolin-4(3H)-one each of which is optionally substituted with one or two substituents independently selected from nitro and alkyl;
hydroxy, hydroxyalkyl, hydroxyalkyloxy, alkyl, carboxy, alkoxy, alkoxyalkyl, alkoxyalkyloxy, —COR [where R is aryl substituted with one halo], -(alkylene)-NRR′ [where R is hydrogen or —COR^{a} (where R^{a} is alkyl), and R′ is hydrogen or alkyl], —O-(alkylene)-NRR′ [where R is hydrogen or —COR^{a} (where R^{a} is alkyl), and R′ is hydrogen or alkyl], —NRR′ [where R is hydrogen or alkyl, and R′ is alkyl, —COR″ (where R″ is alkyl, haloalkyl, or aryl), —SO₂R″ (where R″ is pyridinyl, aralkyl, alkyl, cycloalkyl, or aryl optionally substituted with two alkoxy groups)], piperidin-4-yl-alkyl, piperidin-4-yl, or piperazin-4-yl-alkyl (wherein the piperidinyl in piperidin-4-yl-alkyl or piperidin-4-yl and piperazinyl in piperazin-4-yl-alkyl is substituted with a quinazoline ring optionally substituted with one to three substituents selected from halo, alkyl, alkoxy, haloalkyl, amino, monoalkylamino, or dialkylamino);
2-oxoimidazolidin-1-yl, pyrrolidine-2,5-dione, or 1H-benzo[d]imidazol-2(3H)-one optionally substituted with one alkyl; or
furanylalkyloxy, 3,4-dihydroquinazolin-2(1H)-one, 1,6-alkylquinazoline-2,4(1H,3H)-dione, 1H-benzo[d][1,2,3]triazole, 3,4-dihydrobenzo[e][1,3]oxazin-2-one, 2H-pyran-2-ylalkyloxy, or tetrahydropyrimidin-2(1H)-one-1-ylalkyl, each of which is optionally substituted with alkyl;
(d) imidazolidin-2-one optionally substituted with one alkyl;
(e) piperidin-1-yl, where one of R⁴, R⁵, and R⁶ is hydrogen, the other of R⁴, R⁵, and R⁶ is hydroxyl, and the third of R⁴, R⁵, and R⁶ is alkyl, aralkyl, or aryl, optionally substituted with one or two substitutents independently selected from halo, hydroxyl, or alkoxy;
(f) indol-1-yl substituted with alkyl and alkoxy and a third substituent selected from alkoxycarbonyl or hydroxyalkyl;
(g) aryl substituted with one or two substitutents independently selected from alkoxy, hydroxyl, alkyl, haloalkyl, acetyl, or 4-methylphenylsulfonyl;
(h) piperazin-1-yl substituted with R⁴, R⁵ and R⁶, where two of R⁴, R⁵ and R⁶ are hydrogen and the other of R⁴, R⁵ and R⁶ is acyl; alkyl; aryl optionally substituted with one halo; alkoxycarbonyl; or —CONHR′ (where R′ is aryl optionally substituted with hydroxyl, cyano, nitro, alkyl, or alkylcarbonyl); or morpholin-4-ylcarbonyl;
(i) aryl substituted with R⁴, R⁵, and R⁶, where R⁵ is hydrogen and one of R⁴ and R⁶ is alkyl, halo, amino, nitro, hydroxyl, alkoxy, phenyl, haloalkyl, dialkylamino, or —NHCOR, where R′ is alkyl; and the other of R⁴ and R⁶ is hydrogen, alkyl, amino, or alkoxy; or all R⁴, R⁵, R⁶ are alkoxy; or
(j) 3-halopyridin-4-yl;
(vii) when X and Z or Y and Z are nitrogen, then when two of R¹, R², and R³ are hydrogen, then the other of R¹, R², and R³ is not halo;
(viii) when X and Z are nitrogen, then not all of R¹, R², and R³ are alkoxy; and
(ix) the compound is not a salt of any one (i)-(viii).

2. The compound of claim 1, wherein X and Y are nitrogen and Z is carbon.

3. The compound of claim 1, wherein Y and Z are nitrogen and X is carbon.

4. The compound of claim 1, wherein X and Z are nitrogen and Y is carbon.

5. The compound of any one of claims 2-4, wherein R¹ is hydrogen.

6. The compound of claim 3, wherein R¹ is hydrogen, and R² and R³ are alkoxy.

7. The compound of any one of claims 2-4, wherein R¹ is hydrogen, R² is alkoxy, and R³ is alkyl.

8. The compound of any one of claims 2-4, wherein R¹ is hydrogen, R² is alkyl, and R³ is alkoxy.

9. The compound of claim 1, wherein R^{3a} is a ring of formula (a) 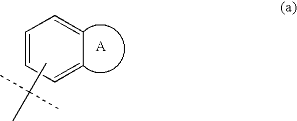
wherein A is a monocyclic five-, six-, or seven-membered heterocyclyl ring substituted with R⁴, R¹ and R⁶.

10. The compound of claim 1, wherein R^{3a} is a ring of formula: 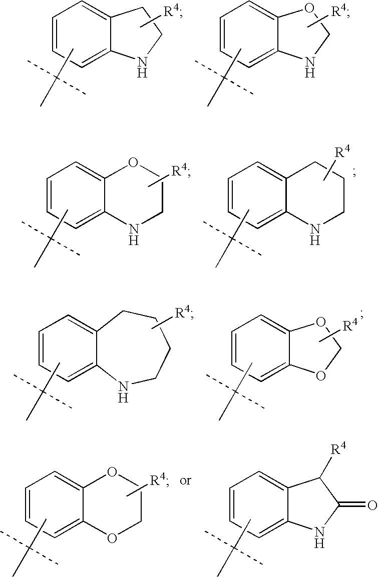
wherein R⁴ is aryl; heteroaryl; heterocyclyl; or —X¹R⁷, where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹² where R⁸-R¹² are independently hydrogen, alkyl, hydroxyalkyl, alkoxyalkyl, aryl, aralkyl, heteroaryl, heteroaralkyl, acyl, or heterocyclylalkyl; and R⁷ is cycloalkyl, cycloalkylalkyl, aryl, heteroaryl, heterocyclyl, aralkyl, heteroaralkyl, or heterocyclylalkyl and optionally substituted, including the ring —NH— group, with R⁵ and R⁶.

11. The compound of claim 1, wherein R^{3a} is a ring of formula: 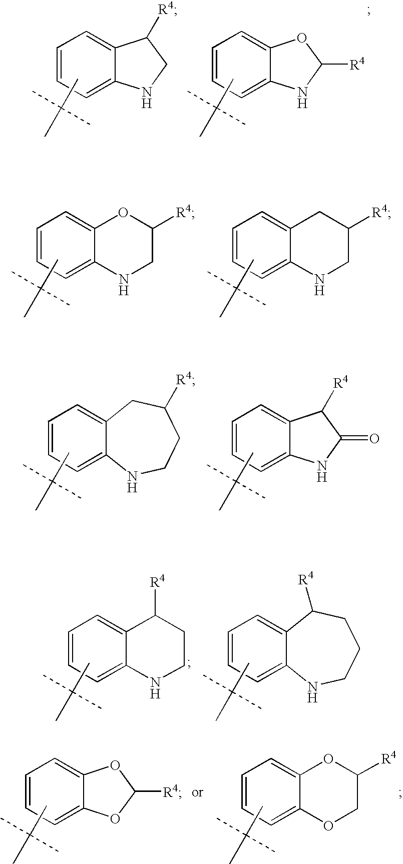
where R⁴ is aryl, heteroaryl, heterocyclyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl and optionally substituted, including the ring —NH— group, with R⁵ and R⁶ as defined above.

12. The compound of claim 1, wherein R^{3a} is a ring of formula: 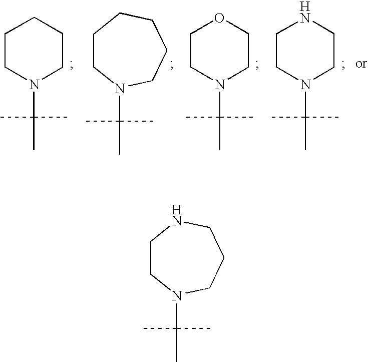
wherein R^{3a} is substituted, including the ring —NH— groups, with R⁴, R⁵ and R⁶.

13. The compound of claim 1, wherein R^{3a} is a ring of formula: 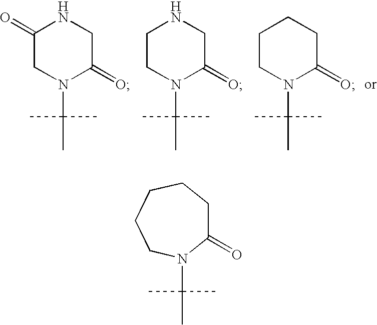
wherein R^{3a} is substituted, including the ring —NH— groups, with R⁴, R⁵ and R⁶.

14. The compound of claim 1, wherein R^{3a} is a ring of formula: 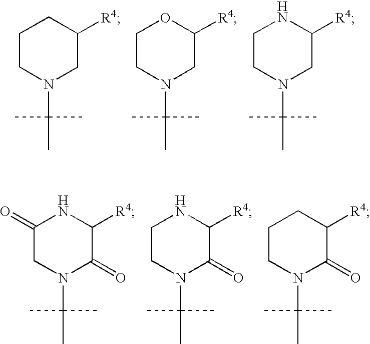
wherein R⁴ is aryl, heteroaryl, or six-membered saturated heterocyclyl, optionally substituted with R^{a}, R^{b} and R^{c}; and wherein the ring is optionally substituted, including the hydrogen atom on the —NH— group within the ring, with R¹ and R⁶.

15. The compound of claim 1, wherein R^{3a} is a ring of formula: 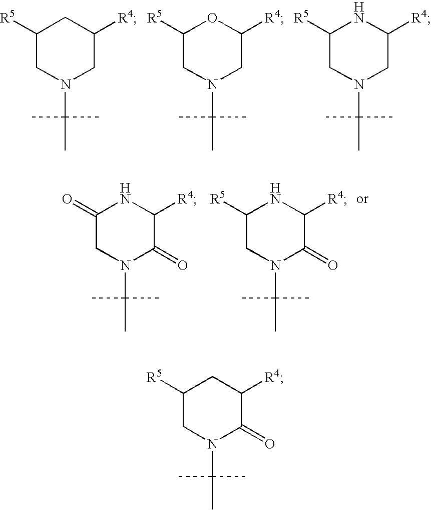
wherein R⁴ is phenyl or heteroaryl, substituted at the para position with R^{a}, and optionally substituted with R^{b} and R^{c}.

16. The compound of claim 1, wherein R^{3a} is a ring of formula: 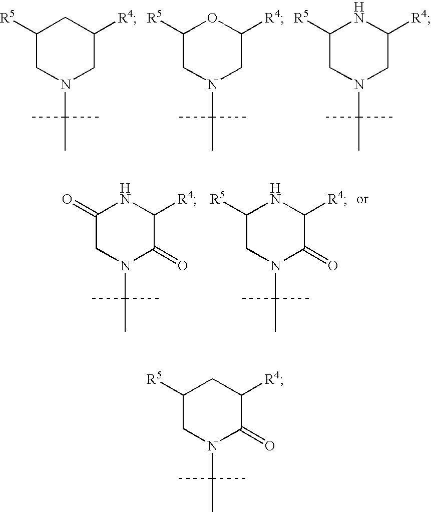
wherein R⁴ is heterocyclyl substituted at the para position with R^{a}, and optionally substituted with R^{b} and R^{c}.

17. The compound of claim 1, wherein R^{3a} is a ring of formula: 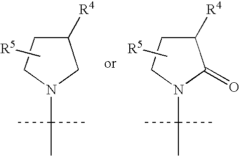
wherein R⁴ is aryl, heteroaryl, or six-membered saturated heterocyclyl, optionally substituted with R^{a}, R^{b} and R^{c}.

18. The compound of claim 1, wherein R^{3a} is a ring of formula: 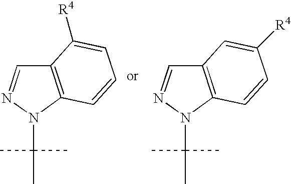
wherein:
R⁴ is aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);
wherein R^{3a} is optionally substituted with R⁵ and R⁶ each independently selected from hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; and
wherein the aromatic or alicyclic ring in R⁴, R⁵, R⁶, and R⁷ is optionally substituted with one to three substitutents independently selected from R^{a}, R^{b}, and R^{c} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino.

19. The compound of claim 18, wherein R⁴ is phenyl, heteroaryl or heterocyclyl.

20. The compound of claim 1, wherein R^{3a} is a ring of formula: 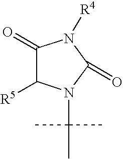
wherein R⁴ is aralkyl, optionally substituted with R^{a}, R^{b} and R^{c}.

21. The compound of claim 1, wherein R^{3a} is a ring of formula: 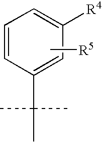
wherein:
one of R⁴ and R⁵ is hydrogen, alkyl, halo, haloalkyl, alkoxy, haloalkoxy, cyano, amino, monsubstituted or disubstituted amino, or —X¹R⁷ (where X¹ is —O—, —CO—, —OC(O)—, —C(O)O, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and
the other of R⁴ and R⁵ is aryl, heteroaryl, or heterocyclyl; and
wherein the aromatic or alicyclic ring in R⁴ and R⁵ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

22. The compound of claim 1, wherein R^{3a} is a ring of formula: 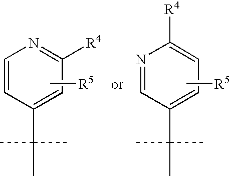
wherein:
one of R⁴ and R⁵ is hydrogen; alkyl; halo; haloalkyl; alkoxy; haloalkoxy; cyano;
amino; monsubstituted or disubstituted amino; or —X¹R⁷ (where X¹ is —O—, —CO—, —OC(O)—, —C(O)O, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and
the other of R⁴ and R⁵ is aryl, heteroaryl, or heterocyclyl; and
wherein the aromatic or alicyclic ring in R⁴ and R⁵ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

23. The compound of claim 1, wherein R^{3a} is a ring of formula: 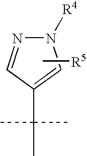
wherein R⁵ is hydrogen or alkyl and R⁴ is aryl, heteroaryl, aralkyl, heteroaralkyl, or heterocyclyl, optionally substituted with one to three substituents independently selected from R^{a}, R^{b}, and R^{e}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

24. A pharmaceutical composition comprising a compound of claim 1 and a pharmaceutically acceptable excipient.

25. A method of treating a disorder treatable by inhibiting a PDE10 enzyme in a patient, wherein the method comprises administering to the patient a pharmaceutical composition comprising a compound of claim 1 and a pharmaceutically acceptable excipient.

26. The method of claim 25, wherein the disorder is schizophrenia, bipolar disorder, or obsessive-compulsive disorder.
